(19) United States
(12) Patent Application Publication
(10) Pub. No.: US 2026/0294307 A1
(43) Pub. Date: Oct. 1, 2026

(54) Patch Device Having Flexible Electrode and Its Operating Method

(71) Applicant: WisMedical Co., Ltd., Yongin-si (KR)

(72) Inventors: Sung Hoon LEE, Seoul (KR); Karam KIM, Tongyeong-si (KR); Woon Hong YEO, Daejeon (KR); Seong Eun KIM, Yongin-si (KR); Joon Yeong KIM, Yongin-si (KR); Soo Jin LEE, Gimhae-si (KR)

(21) Appl. No.: 19/305,032

(22) Filed: Aug. 20, 2025

(30) Foreign Application Priority Data
Mar. 25, 2025 (KR) 10-2025-0038257

(51) Int. Cl. A61B 5/257; A61B 5/00; A61B 5/11; A61B 5/1455; A61B 5/263; A61B 5/273; A61B 5/282; A61B 5/291; A61B 5/296; A61B 5/297; A61B 7/00

(52) U.S. Cl. CPC A61B 5/257; A61B 5/0022; A61B 5/1126; A61B 5/14552; A61B 5/263; A61B 5/273; A61B 5/282; A61B 5/291; A61B 5/296; A61B 5/297; A61B 5/7264; A61B 7/006; A61B 2562/166

(57) ABSTRACT

The patch device includes at least one electrode for acquiring a user biometric signal, a conductive layer connected to each of the at least one electrode, a connector connected to the conductive layer and configured to transmit the user biometric signal acquired from the at least one electrode to at least one entity within the patch device, and at least one entity configured to receive the user biometric signal and transmit it to a device. Each of the at least one electrode may include a fabric layer that maintains a non-adhesive state, an adhesive layer located below the fabric layer and attachable to the user's body, a flexible electrode within the adhesive layer for sensing the user biometric signal from the user's body, and a silicone adhesive portion bonded to the entire electrode, wherein the flexible electrode may have a serpentine pattern.

### TECHNICAL FIELD

The present invention relates to a patch device having a flexible electrode and a method for operating the same. More particularly, it relates to a patch device that is attachable to a user's body to acquire biometric signals from the user, and to a method for operating such a device.

### BACKGROUND ART

Conventional wearable devices (or medical devices) and systems have been used to monitor a user's pulse or heart rate to assess the user's health condition and provide feedback information, enabling the user to adjust exercise intensity accordingly. These conventional wearable devices could detect user movement to measure step count, calories burned, and other metrics, thereby allowing users to monitor their physical activity. Especially during activities such as running, cycling, and swimming, users could check measurement data from the wearable device.

However, existing wearable devices and systems have limitations in acquiring detailed physical (or biometric) information of users. Specifically, these devices cannot obtain biometric signals that vary according to internal physiological changes. Given increasing concerns about real-time health monitoring, there is a growing need to measure and analyze biometric signals such as fatigue, stress, or reactions to external stimuli. Existing devices have not fully addressed this need.

In view of the above, the present disclosure describes a patch device and method that can be attached to the user's body to monitor biometric signals in real time. This patch device may be referred to as a medical device, wearable device, or otherwise, and is not limited to a specific form. When attached to the user's body, the patch device must acquire highly accurate signals. If the device is not securely attached or detaches due to user movement, signal acquisition may be impaired. Solutions to these issues are presented herein.

### PRIOR ART LITERATURE

### Patent Literature

(Patent literature 0001) Korean Patent Registration Publication No. 10-2377703

### SUMMARY

### Problems to be Solved

The present disclosure relates to a patch device having a flexible electrode and a method for operating the same.

It provides a method and device for acquiring biometric signals from a user using a patch device equipped with a flexible electrode.

It also discloses a patch device having a serpentine-patterned flexible electrode capable of maintaining mechanical coupling while ensuring electrical stability.

Furthermore, the invention provides a method and device for analyzing a user's biometric signals using an AI (Artificial Intelligence) or ML (Machine Learning) model trained based on signals acquired from at least one patch device equipped with flexible electrodes.

### Means for Solving the Problems

According to one embodiment of the present disclosure, a patch device includes: at least one electrode for acquiring a user's biometric signal; a conductive layer connected to each of the at least one electrodes; a connection unit for transmitting the biometric signal acquired from the electrode via the conductive layer to at least one entity within the patch device; and at least one entity that receives the biometric signal and transmits it to an external device. Each of the at least one electrodes may comprise: a fabric layer maintaining a non-adhesive state; an adhesive layer positioned below the fabric layer and attachable to the user's body; a flexible electrode embedded within the adhesive layer for sensing the user's biometric signal from the body; and a silicone adhesive portion that covers the entirety of the electrode. The flexible electrode may have a serpentine pattern.

According to another embodiment of the present disclosure, a system including at least one patch device is provided. The system includes: at least one patch device; a device connected to the patch device for controlling the same; and a cloud that receives the biometric signal from the device to perform user monitoring and generate analysis information. Each of the patch devices may include: at least one electrode for acquiring a user's biometric signal; a conductive layer connected to each of the at least one electrodes; a connection unit for transmitting the signal to at least one entity within the patch device; and at least one entity for delivering the signal to the external device. Each electrode may comprise a fabric layer maintaining a non-adhesive state; an adhesive layer located beneath the fabric layer and attachable to the user's body; a flexible electrode for sensing biometric signals embedded in the adhesive layer; and a silicone adhesive portion surrounding the entire electrode. The flexible electrode may be formed in a serpentine pattern.

In addition, the following features may commonly apply:

According to one embodiment, the electrode may be an ENIG (Electroless Nickel Immersion Gold) electrode composed of an electroless nickel layer formed on copper to prevent direct contact between copper and gold, and an immersion gold layer formed as a thin gold coating on the nickel. The immersion gold layer may contact the user's body to acquire the biometric signal.

According to another embodiment, a polyimide layer may be interposed between conductive layers to improve mechanical performance. The connection unit may include at least one flat flexible cable (FFC) connector connected to each electrode, or pogo pins to which the electrodes are connected.

According to another embodiment, the adhesive layer of the patch device may have a predefined pattern. When the user's body to which the adhesive layer is attached contracts or expands, the adhesive layer deforms accordingly via the pattern to maintain mechanical bonding to the body surface. The predefined pattern may be a kirigami pattern. When the user body expands, the adhesive layer may expand along the kirigami pattern, increasing surface area to maintain contact. When the user body contracts, the layer may contract accordingly, increasing adhesive force to maintain mechanical coupling.

According to another embodiment, the patch device may be a heart patch comprising a plurality of electrodes. The heart patch may acquire one or more of an ECG (electrocardiogram), SCG (seismocardiogram), or PCG (phonocardiogram) signal as a biometric signal. If the device is a heart patch, it may also include a microphone for acquiring heart sound data.

According to another embodiment, the patch device may be a head patch comprising a plurality of electrodes. The head patch may acquire EEG (electroencephalography) and EOG (electrooculography) signals through the a plurality of electrodes, and may further acquire oxygen saturation data using a PPG (photoplethysmography) sensor.

According to another embodiment, the patch device may be a muscle patch comprising six electrodes. The muscle patch may acquire EMG (electromyography) signals through the plurality of electrodes, and obtain user motion data using a motion sensor. If the patch device is a muscle patch, it may further include a PPG sensor for acquiring oxygen saturation data.

According another embodiment, the patch device may include at least one of a heart patch, head patch, or muscle patch. The patch device may acquire biometric signals and transmit them to a device, and the biometric signals may be further transmitted from the device to a cloud system for analysis and diagnosis.

According to another embodiment, the biometric signals may be transmitted from the device to the cloud to generate user monitoring and analysis information. The cloud may include a learning model, and the user monitoring and analysis information may be provided as input to the model, from which inference results are generated.

### Effects of the Invention

The present disclosure provides a patch device comprising a flexible electrode and a method for operating the same.

The present disclosure offers a method for acquiring a user's biometric signal using a patch device equipped with a flexible electrode.

The present disclosure provides a method for configuring the flexible electrode within the patch device.

The present disclosure provides a method for maintaining mechanical coupling while ensuring electrical stability through a patch device having a flexible electrode with a serpentine pattern.

The present disclosure enables the training of AI/ML (Artificial Intelligence/Machine Learning) models based on signals acquired from at least one patch device comprising a flexible electrode, and provides a method for analyzing the user's biometric signals based on the trained models.

The technical problems addressed by the present disclosure are not limited to the descriptions above and may be further derived or expanded through various embodiments described below.

### BRIEF DESCRIPTION OF THE DRAWINGS

FIG. 1 is a diagram illustrating an example of an operating environment of a system according to an embodiment of the present disclosure.

FIG. 2 is a block diagram illustrating the internal configuration of a computing device (200) according to an embodiment of the present disclosure.

FIGS. 3A, 3B and 3C are diagrams illustrating respective patch devices according to an embodiment of the present disclosure.

FIGS. 4A, 4B and 4C are diagrams illustrating the shapes of the respective patches according to an embodiment of the present disclosure.

FIG. 5 is a diagram illustrating a flexible electrode of the patch device according to an embodiment of the present disclosure.

FIGS. 6A, 6B, 6C, 6D, 6E and 6F are diagrams illustrating an electrode of the patch device according to an embodiment of the present disclosure.

FIGS. 7A, 7B, 7C, 7D, 7E and 7F are diagrams illustrating another electrode configuration of the patch device according to an embodiment of the present disclosure.

FIGS. 8A, 8B, 8C, 8D, 8E and 8F are another diagrams illustrating an electrode configuration of the patch device according to an embodiment of the present disclosure.

FIGS. 9 A and 9B are diagrams illustrating a connection version of the patch device according to an embodiment of the present disclosure.

FIGS. 10 A and 10B are another diagrams illustrating a connection version of the patch device according to an embodiment of the present disclosure.

FIG. 11 is a diagram illustrating the device structure of the patch device according to an embodiment of the present disclosure.

FIG. 12 is a diagram illustrating a system including at least one patch device according to an embodiment of the present disclosure.

FIGS. 13 A and 13B are views illustrating a method of manufacturing a flexible circuit substrate applicable to the present disclosure.

FIGS. 14 A, 14 B and 14C illustrate configurations related to a patch device housing applicable to the present disclosure.

FIG. 15 is views illustrating structures of a patch device applicable to the present disclosure.

FIG. 16 is views illustrating structures of a patch device applicable to the present disclosure.

### DETAILED DESCRIPTION OF EXEMPLARY EMBODIMENTS

In describing the embodiments of the present disclosure, detailed descriptions of publicly known configurations or functions are omitted if it is determined that such descriptions would obscure the essence of the embodiments. In the drawings, parts unrelated to the description of the embodiments are omitted, and similar parts are designated with like reference numerals.

In the embodiments of the present disclosure, when an element is described as being “connected,” “coupled,” or “joined” to another element, it may include both direct and indirect connections through one or more intervening elements. Additionally, when an element is described as “including” or “having” another element, unless otherwise specified, it is understood to include the possibility of additional elements, not the exclusion thereof.

The terms “first,” “second,” etc., in the embodiments are used merely to distinguish one component from another and do not imply any order or importance. For example, a first component in one embodiment may be referred to as a second component in another embodiment and vice versa.

Distinct components described in the embodiments are for clearly explaining their characteristics and do not necessarily mean that they are physically or functionally separate. Multiple components may be integrated into one hardware or software unit, or a single component may be divided into multiple units. Integrated or distributed configurations are also included within the scope of the embodiments.

The term “network” in the present disclosure may encompass both wired and wireless networks, and refers to any communication system through which data may be exchanged between devices and systems. It is not limited to a specific network type.

The embodiments described herein may be implemented entirely in hardware, or as a combination of hardware and software, or entirely in software. Terms such as “unit,” “device,” or “system” refer to entities including hardware, a combination of hardware and software, or software alone. For example, a module, unit, device, or system may be a running process, processor, object, executable, execution thread, program, and/or computer. This is not limiting, and the term may include applications and the devices executing them.

The term “device” in this disclosure may include mobile devices such as smartphones, tablet PCs, wearable devices, HMDs (Head Mounted Displays), as well as stationary devices such as PCs and consumer electronics with display functions. A device may also include vehicle-mounted clusters or IoT (Internet of Things) devices. Thus, a “device” refers to any equipment capable of running an application.

The communication method of the network is not limited. Different components may be connected by different types of network methods. The term “network” may refer to communication methods including telecommunications networks (e.g., mobile networks, wired/wireless Internet, broadcast networks, satellite networks) and short-range wireless communications. It includes all methods by which objects can communicate, including but not limited to wired, wireless, 3G, 4G, 5G, LAN, MAN, GSM, EDGE, HSDPA, W-CDMA, CDMA, TDMA, Bluetooth, Zigbee, Wi-Fi, VoIP, LTE Advanced, IEEE802.16m, WirelessMAN-Advanced, HSPA+, 3GPP LTE, Mobile WiMAX, UMB, Flash-OFDM, iBurst, MBWA, HIPERMAN, BDMA, WiMAX, and ultrasonic communication.

The components described in various embodiments are not necessarily essential and some may be optional. Embodiments formed by a subset of components described are also included within the scope of the invention. Embodiments that include additional components beyond those described are also within the scope.

Hereinafter, the embodiments will be described in detail with reference to the drawings.

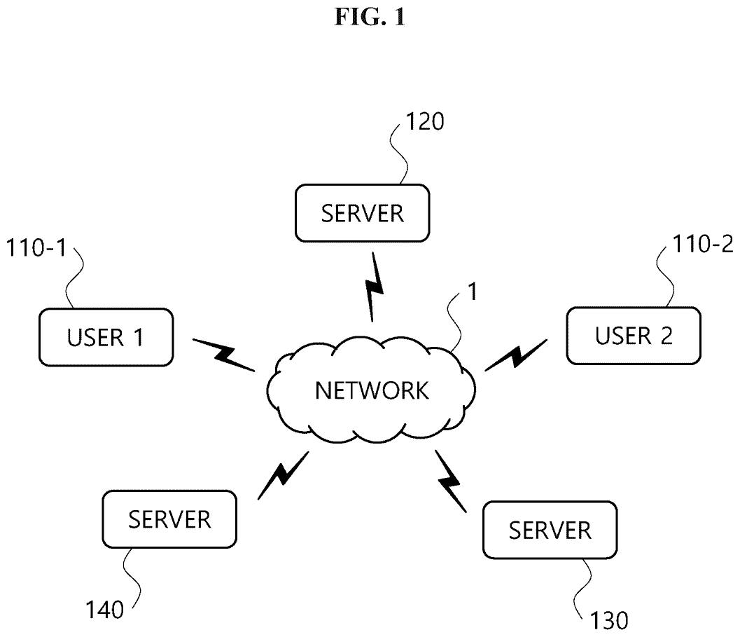

FIG. 1 illustrates an example of an operating environment of a system according to one embodiment. As shown, one or more user devices (110-1, 110-2) and one or more servers (120, 130, 140) are connected via a network (1). The number of devices and servers shown in FIG. 1 is illustrative and not limiting.

Each user device (110-1, 110-2) may be a fixed or mobile terminal implemented as a computer system. Examples include smartphones, mobile phones, navigation devices, computers, laptops, digital broadcasting terminals, PDAs, PMPs, tablet PCs, game consoles, wearable devices, IoT devices, VR devices, and AR devices. In the embodiments, the user device (110) may be any physical computer system capable of communicating with servers (120-140) via the network (1) using wired or wireless communication.

Each server may be a computer or a plurality of computers that communicate with one or more user devices (110-1, 110-2) over the network (1) and provide commands, code, files, content, and services. For example, a server may be a system that provides respective services to one or more user devices (110-1, 110-2) connected through the network(1). More specifically, the server may be a computer program installed and executed on one or more user devices (110-1, 110-2), and may provide a desired service (e.g., information delivery) through an application. In another example, the server may distribute installation and execution files for the aforementioned application to one or more user devices (110-1, 110-2), and receive user input to deliver a corresponding service.

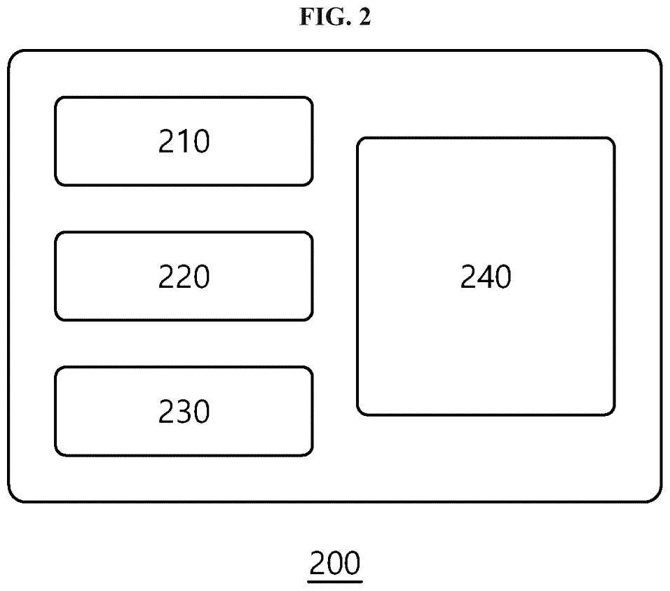

FIG. 2 is a block diagram illustrating the internal structure of a computing device (200) according to one embodiment. The computing device (200) may be any of the user devices (110-1, 110-2) or servers (120-140) described above. Devices and servers may have similar or identical structures with modifications.

As shown in FIG. 2, the computing device (200) may include a memory (210), a processor (220), a communication module (230), and a transceiver (240). The memory (210) is a non-transitory computer-readable medium, such as RAM, ROM, disk drives, SSDs, or flash memory. Non-volatile mass storage devices may also be separate from the memory (210) and part of the device or server. The memory (210) may store an operating system and at least one program code, such as a browser or application installed on a user device (110). These software components may be loaded from separate computer-readable media, such as floppy disks, tapes, DVDs/CD-ROMs, or memory cards.

In another embodiment, the software may be loaded into memory (210) via the communication module (230), such as from a file distribution system or server over the network (1).

The processor (220) performs arithmetic, logic, and I/O operations to process instructions from memory (210) or communication module (230). It may execute commands based on program code stored in memory.

The communication module (230) provides communication functions for devices (110) and/or servers (120-140) via the network (1) and may enable communication with other electronic devices.

The transceiver (240) may serve as an interface with external input/output devices. Input devices may include keyboards, mice, microphones, and cameras. Output devices may include displays, speakers, and haptic feedback devices. A touchscreen may serve as both input and output.

Depending on the nature of the device, computing device (200) may include more components than shown in FIG. 2. If applied to a user device (110), it may include at least some I/O devices, transceivers, GPS modules, cameras, various sensors, databases, and more. In a smartphone, for instance, components may include accelerometers, gyroscopes, camera modules, physical buttons, touch panels, I/O ports, and vibrators.

The patch device with a flexible electrode described below may be a device that communicates with other devices, systems (or servers), and other components based on the network of FIG. 1. The patch device with a flexible electrode may operate based on the computing device (200) of FIG. 2, but is not limited thereto. The patch device with a flexible electrode may also operate within a system comprising at least one patch device. For example, a system comprising at least one patch may include at least one patch device with a flexible electrode, a device for processing signals acquired from the patch device, and a cloud (or server) for analyzing signals delivered from the device, any one or more of which may be included. The patch device, device, and cloud may operate based on the computing device (200) of FIG. 2.

More specifically, each patch device may be a low-power device including only part of the configuration of the computing device (200) of FIG. 2. Here, each device included in the system may exchange signals or data based on the network shown in FIG. 1, although the invention is not limited to that embodiment. For instance, biometric signals sensed from at least one patch device may be transmitted to a device. The device may analyze the biometric signal information acquired from at least one patch device and provide it to a user or transmit it to the cloud for analysis.

The system may be composed of only at least one patch device and a device, while the cloud or server may be located externally. In addition, the patch device with a flexible electrode may operate as a standalone device and interact with an external system or device. That is, the patch device with a flexible electrode may be included in a specific system or operate as an independent device and is not limited to any specific form. For convenience, the following description is based on a case where the patch device with a flexible electrode interacts with a device and a cloud, but it is not limited thereto.

The patch device with a flexible electrode may include at least one of a heart patch, head patch, or muscle patch. The patch device with a flexible electrode may be a patch of another form, but is not limited thereto. Here, the heart patch may be a patch for measuring an ECG (Electrocardiogram) signal, the head patch may be a patch for measuring an EEG (Electroencephalogram) signal, and the muscle patch may be a patch for measuring an EMG (Electromyography) signal. The patch device with a flexible electrode may be attached to the user's body as one of the above-mentioned patches and acquire the corresponding signal from the user. In addition, the patch device with a flexible electrode may be a patch device operating within the system, and the signals acquired from the user's body via at least one patch device may be transmitted to the device and utilized for user monitoring and diagnosis.

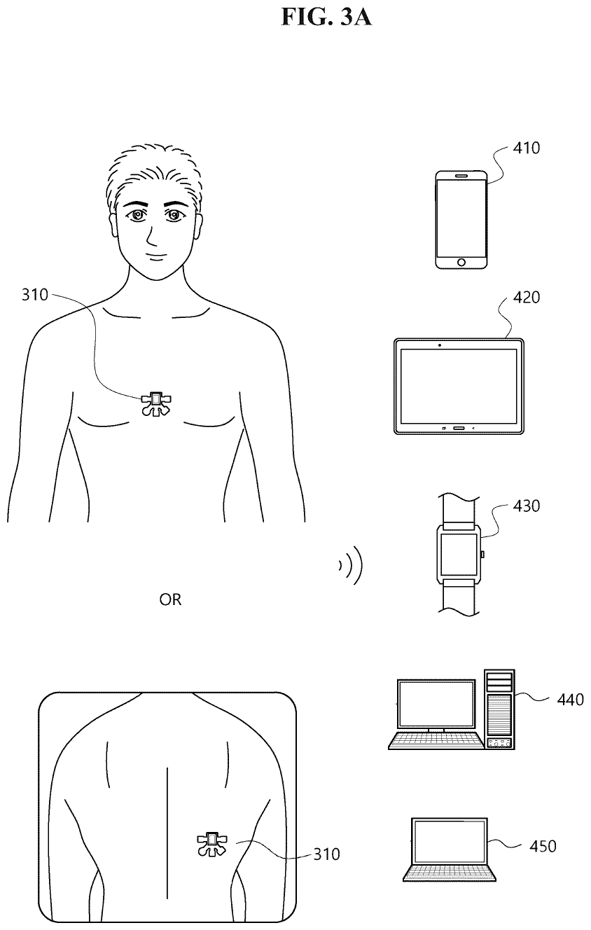

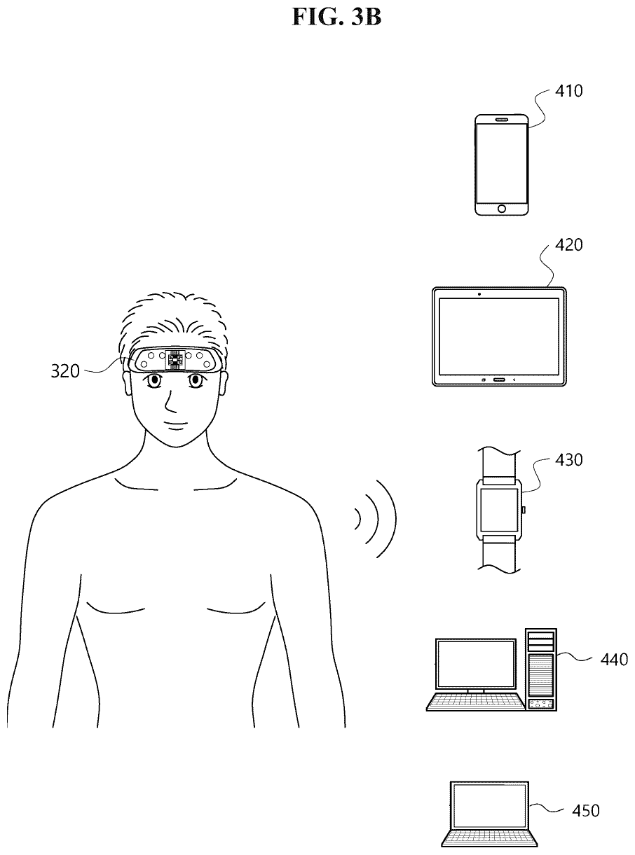

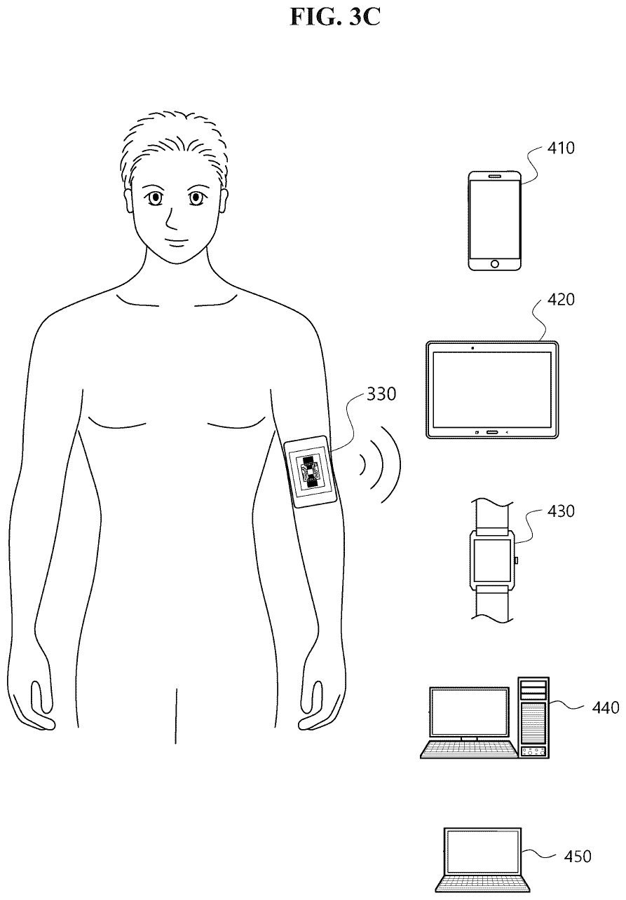

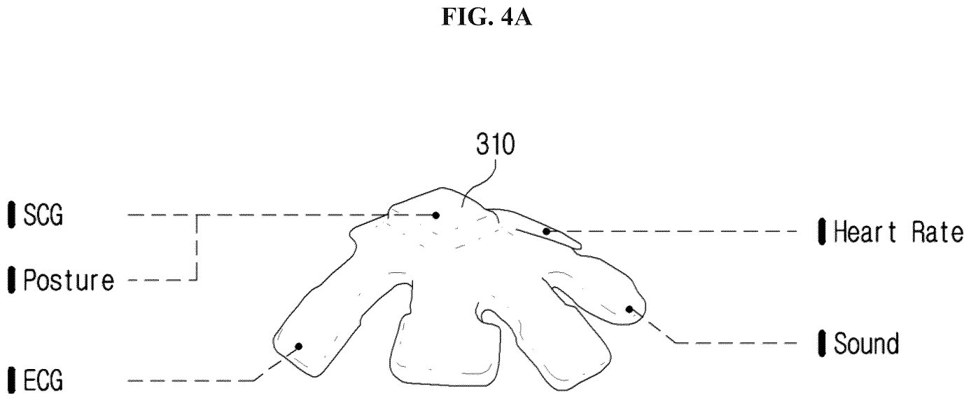

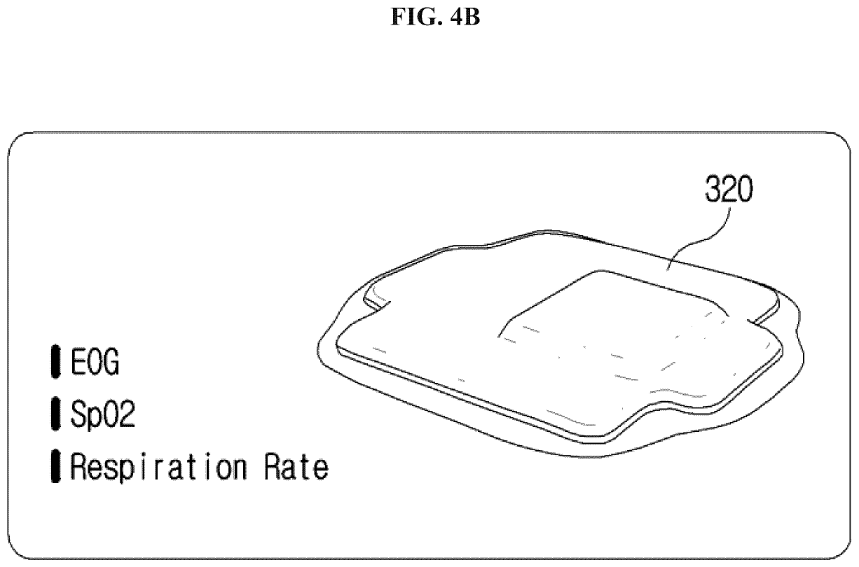

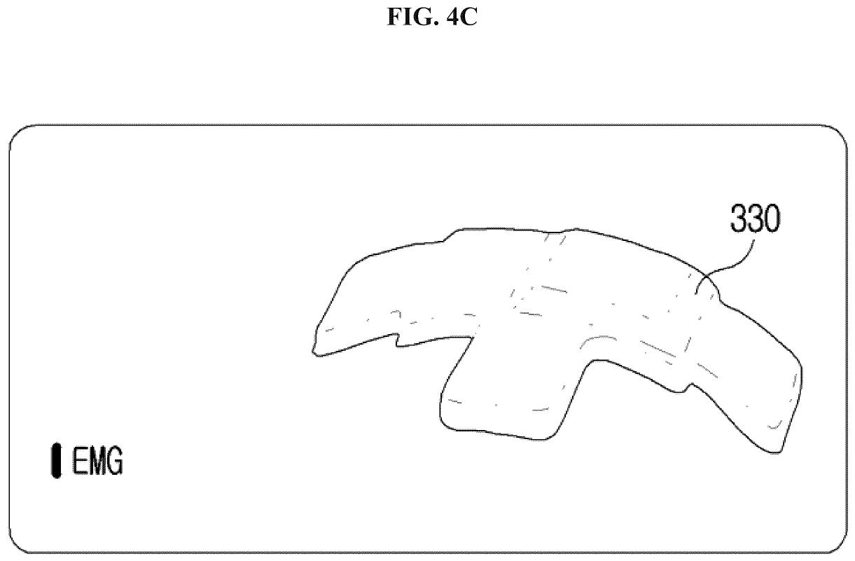

FIGS. 3a to 3c illustrate respective patch devices according to an embodiment of the present disclosure. Additionally, FIG. 4 illustrates the shape of each patch according to an embodiment.

In FIGS. 3a to 3c, the patch devices may transmit sensed signals to a user device. Here, the user device may include at least one of a smartphone (410), smart pad (420), smartwatch (430), PC (440), or laptop (450). The user device may be a device that receives biometric signals sensed by each patch device and is not limited to the above-mentioned devices.

In one embodiment, the user device may be a smartphone (410) carried by the user, and each patch device may transmit the sensed biometric signals to the smartphone (410) in real time. The user may monitor the sensed biometric signals in real time.

In another embodiment, the user device may be a PC (440). Each patch device may be attached to the user's body and used in a medical institution (e.g., sleep center) or at the user's home, and transmit the sensed signals to the PC (440). That is, the patch devices may be used in a designated environment to sense the user's biometric information, and a PC (440) may be used as the corresponding user device.

When each patch device is used for real-time monitoring through a mobile user device, the software (or application, interface) used on the mobile device and that on the PC may differ. However, the invention is not limited to this embodiment. For convenience, the following description uses a portable user device as a reference, but is not limited to this form.

Referring to FIG. 3a, the heart patch (310) may be attached to the body surface where the user's heart is located. The heart patch (310) may be attached to the body and may acquire heart-related signals. Here, the heart-related signals acquired by the heart patch (310) may include at least one of ECG (electrocardiogram), SCG (seismocardiogram), and PCG (phonocardiogram). ECG may be an electrical signal related to heartbeats, SCG may be a low-frequency vibration signal of the heart, and PCG may be a heart sound signal. That is, the heart patch (310) may acquire at least one or more of electrical signals, vibration signals, and acoustic signals related to heartbeats. In addition, the heart patch (310) may further include at least one of posture information, heart rate information, and heart sound information. That is, the heart patch (310) may detect changes in the user's posture, obtain heart rate information based on user activity, and obtain heart sound information. The heart patch (310) may have a shape as shown in FIG. 4A, and based on such a shape, the heart patch (310) may be attached to the user's body. However, FIG. 4A is merely an example of the shape of the heart patch (310), and the shape is not limited thereto.

In one embodiment, the heart patch (310) may be attached to the front of the user's body at the heart location to acquire heart-related signals. The heart-related signals may include the information described above. In another example, the heart patch (310) may be attached to the back (dorsal side) of the user's body at the heart location to acquire heart-related signals. In another example, multiple heart patches (310) may be attached to the body. Specifically, the heart patch (310) may be attached to both the front and back sides of the user's body at the heart location. Heart-related signals may be acquired from each of the plurality of heart patches (310) and compared, and based on the compared signals, heart-related signals may be acquired, thereby improving the accuracy of heart-related signal measurement.

In another embodiment, the heart patch (310) may acquire additional information related to heartbeats or the user's body. The additional information may include the aforementioned posture information. Alternatively, the heart patch (310) may acquire the user's body temperature or skin moisture content, which may be the aforementioned additional information. The user's body temperature or skin moisture content may be information that affects the acquisition of heart-related signals by the heart patch (310), and the heart patch (310) may further acquire such information. In a specific example, in cold weather such as winter, if the user's body temperature decreases, the heartbeat may slow down, and the heart patch (310) may further acquire information such as body temperature, external temperature, and other data. In another example, the heart patch (310) may further acquire location information, environmental information, and other data. Specifically, the heart-related signal patterns of a user using the heart patch (310) may differ depending on whether the user is indoors or outdoors. Alternatively, heart-related signals may differ between a situation where the user is at home feeling psychologically relaxed and a situation where the user is at an office performing work. Here, the heart patch (310) may measure the user's location information together and transmit it to the user device. In another example, the heart patch (310) may acquire environmental information, which may be information about the surroundings of the user. For example, the environmental information may include temperature, humidity, wind strength, and other information that may affect heartbeats. That is, the heart patch (310) may acquire additional information related to heartbeats, but is not limited thereto.

Thereafter, the heart patch (310) may transmit the acquired signals described above to the user device. The user device may be a device used by the user wearing the heart patch (310) and may be one of a smartphone (410), tablet (420), smartwatch (430), PC (440), laptop (450), or other devices, and is not limited to a specific type. However, for convenience of description, the tablet (420) among the user devices is used as an example in the following description, but it is not limited thereto. Thereafter, the user device may perform monitoring based on the signals acquired from the heart patch (310). Here, the user device may transmit the signals acquired from the heart patch (310) to a cloud or server.

In another embodiment, the user device may be a device that operates within a preset distance from the heart patch (310). In a specific example, the user device may be a smartwatch (430). Here, the user device may acquire the aforementioned location information and environmental information within a preset distance from the heart patch (310), and is not limited to the above-described embodiment. For example, the smartwatch (430) among the user devices may measure the location information and environmental information within a preset distance from the heart patch (310) and transmit them to another user device (e.g., tablet 420). That is, the tablet (420) may acquire heart-related signals from the heart patch (310) and further acquire user-related additional information through the smartwatch (430), but the embodiment is not limited thereto.

Referring to FIG. 3b, the patch device may be a head patch (320). The head patch (320) may be attached to the user's body at the head (or forehead) position and may acquire brainwave and other related signals. The brainwave and other related signals acquired by the head patch (320) may include at least one of EEG (Electroencephalography), EOG (Electrooculography), and oxygen saturation. Additionally, the head patch (320) may further measure respiration rate information and other signals.

EEG may be a signal related to the electrical activity of the brain and may include signals across various frequency bands generated by the brain. Specifically, signals generated in the user's brain may differ depending on the user's activity or movement, and the different signals may have different frequencies. For example, EEG may include at least one of delta, theta, alpha, beta, and gamma signals. Furthermore, EEG may include additional signals and is not limited to specific forms. The head patch (320) may measure EEG generated in the user's brain in real time and utilize the measured signal. The head patch (320) may also measure EOG by detecting electrical potential across the eye. EOG refers to a technique for measuring the potential between the cornea and retina to detect eye movement. Specifically, EOG may measure eye movement based on the potential difference between the front and rear of the eye. The head patch (320) may measure EOG signals in real time and utilize the measured signals. Additionally, the head patch (320) may measure oxygen saturation, which may be the oxygen concentration in the blood. The head patch (320) may acquire oxygen saturation signals. In a specific example, the head patch (320) may include a light source and a photosensor and may acquire oxygen saturation information in the blood by measuring infrared and visible light using the photosensor. However, this embodiment is not limited to this configuration. The head patch (320) may measure oxygen saturation-related signals in real time and utilize the measured signals. The head patch (320) may be attached to the forehead position on the front of the user's face to acquire the above EEG, EOG, and oxygen saturation signals. Additionally, the head patch (320) may further acquire the user's respiration rate information. The head patch (320) may acquire other signals as well and is not limited to specific signal types. The head patch (320) may have a shape as shown in FIG. 4B, and may be attached to the user's body based on that shape. However, FIG. 4B is only one example of the head patch (320) and is not limited to that particular shape.

The head patch (320) may be attached to the forehead of the user's face to acquire head-related signals. In another example, the head patch (320) may be configured in the form of a sleep pad and be attached to the eye area of the user's face. In yet another example, the head patch (320) may be attached to the user's body in conjunction with a hat, headband, or other wearable device and acquire head-related signals. It is not limited to any particular form.

In another embodiment, multiple head patches (320) may be attached to the body. The head patch (320) may be attached to the front, back, and sides of the body. The head-related signals acquired from each of the multiple head patches (320) may be compared to each other, and such comparison may increase the measurement accuracy of the head-related signals. However, this is not limited to such configurations. For convenience of description, the following examples are based on the configuration where the head patch (320) is attached to the forehead on the front of the user's face, but it is not limited to that configuration.

Additionally, the head patch (320) may acquire additional information besides brainwave-related signals. For example, the head patch (320) may further acquire the user's body temperature or skin moisture information. The user's body temperature or skin moisture may affect the acquisition of head-related signals when using the head patch (320), acting as interfering signals. Thus, the head patch (320) may acquire such information to increase the measurement accuracy of head-related signals. In a specific example, in the summer, the user's body temperature may increase, and the surface may contain more moisture due to sweat. In such cases, signal correction may be necessary. The head patch (320) may further acquire such information to improve the measurement accuracy of head-related signals.

Referring to FIG. 3c, the muscle patch (330) may be a patch device that is attached to the user's body to acquire EMG signals. The muscle patch (330) may sense EMG (Electromyography), which may be electrical signals generated by muscle activity. Specifically, the muscle patch (330) may detect electrical signals generated within muscle fibers due to muscle contraction/relaxation and may sense EMG signals generated by the detected electrical signals. Muscles of the body may contract and relax in response to signals delivered from the nervous system, and EMG signals may be generated by such signals. EMG signals may be measured through electrodes attached to the skin surface. In one embodiment, the acquired EMG signals may be analyzed based on amplitude and frequency information, and the analysis of such information may be used for monitoring the user's sleep state and sleep diagnosis. The muscle patch (330) may have a shape as shown in FIG. 4C, and based on that shape, it may be attached to the user's body. However, FIG. 4C is merely one example of the muscle patch (330), and the shape is not limited thereto.

In one embodiment, the muscle patch (330) may be attached to the user's arm to acquire EMG signals from the user's arm. The muscle patch (330) attached to the arm may sense the user's movement and acquire sensed movement information. For example, the user's movement information may be information related to motion during a static state. Specifically, the movement information may include sleep pattern information such as tossing or slight movement during sleep. The movement information may be based on a preset cycle, preset pattern, or compared to previously stored information, but is not limited to a specific form. The muscle patch (330) may acquire various types of user movement information and is not limited to any specific type. Additionally, the muscle patch (330) may acquire the user's heart rate, oxygen saturation, body temperature, blood pressure, respiration rate, and other information, and is not limited to a specific type. In another example, the muscle patch (330) may be attached to the user's jaw. The muscle patch (330) may be attached to the user's jaw and may acquire EMG signals based on electrical signals generated by jaw movement. In a specific example, the muscle patch (330) included in a sleep diagnosis system may be attached to the user's jaw to measure EMG signals. The muscle patch (330) may acquire signals related to jaw or facial movement during sleep and may perform sleep pattern or sleep analysis based on such data. In another example, the muscle patch (330) may be attached to the user's jaw to acquire other types of signals, and is not limited to a specific form.

The muscle patch (330) may be attached to the user's arm to acquire patch-related signals related to a sleep diagnosis system. In another embodiment, the muscle patch (330) may be attached to another part of the user's body to acquire patch-related signals. In one embodiment, the muscle patch (330) may sense the attachment location on the user's body and may sense patch-related signals based on the attached location. If the muscle patch (330) is attached to the left arm, it may recognize the user's left arm and acquire patch-related signals considering the body part. If the muscle patch (330) is attached to the right arm, it may recognize the right arm and acquire signals accordingly.

In another embodiment, multiple muscle patches (330) may be simultaneously attached to different parts of the user's body. Specifically, depending on the sleep diagnosis system including the muscle patch (330), it may be necessary to acquire patch-related signals from multiple body parts, and considering this, the muscle patch (330) may be attached to multiple body parts. Here, the muscle patches (330) attached to multiple body parts or other patches included in the sleep diagnosis system may interact with each other, and based on this, signals related to sleep monitoring and sleep diagnosis may be acquired. In another embodiment, the muscle patch (330) may further acquire additional information besides patch-related signals. For example, the muscle patch (330) may acquire the user's body temperature or skin moisture information. The body temperature or skin moisture of the user may be information that may affect patch-related signal acquisition by acting as interference, and the muscle patch (330) may further acquire such information to improve patch signal measurement accuracy.

Additionally, each of the patches in FIGS. 3a to 3c may be a beacon device. Alternatively, each of the patches in FIGS. 3a to 3c may be a low-power device other than a beacon. A beacon device may be a small device that transmits and receives data at short distances using short-range wireless communication technology and may operate based on Bluetooth Low Energy (BLE) technology. Each of the patches in FIGS. 3A to 3C may be implemented as a beacon device and may transmit signals sensed from the user's body to a user device (400) based on this. In another example, each of the patches in FIGS. 3a to 3c may be a different type of low-power device and is not limited to a specific form.

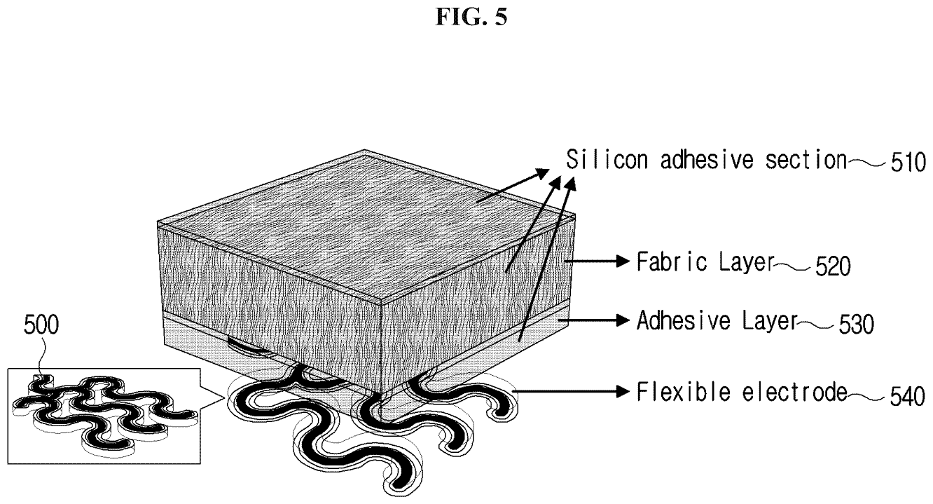

FIG. 5 is a diagram illustrating a flexible electrode of the patch device according to an embodiment of the present disclosure.

As described above, the patch device may be configured in various forms and may be attached to the user's body to acquire biometric signals sensed from the user. For example, the biometric signals may refer to signals acquired from the user by the above-described heart patch (310), head patch (320), muscle patch (330), and other patches, and are not limited to specific types of signals. That is, the biometric signals may refer to signal information acquired from the user via the patch device and may exist in various forms. For convenience of explanation, these are referred to as biometric signals.

Since the patch device senses biometric signals while attached to the user's body, it needs to be closely adhered to the body. If the patch device is not closely adhered to the user's body or is partially detached due to user activity, the biometric signals acquired from the user may differ. Specifically, signals sensed while the patch device is in close contact with the skin may differ from signals sensed when part of the patch is detached. That is, changes in the mechanical coupling between the patch and the user's body can result in differences in the sensed signals. In another embodiment, if there are foreign substances on the electrode sensing the biometric signals, electrical changes may occur, and the sensed signals may vary accordingly. This could reduce the accuracy of biometric signal acquisition.

In view of the above, the patch device preferably maintains contact with the user's body in a clean state without foreign substances. That is, the degree of mechanical coupling between the patch and the user's body should be consistently maintained, and a method to achieve this is required.

The patch device may be provided with a flexible electrode. The patch device may include at least one electrode, and each of the electrodes may be attached to the body to acquire the aforementioned biometric signals. Each sensing electrode may detect changes in electrical signals and sense biometric signals accordingly. The biometric signals obtained from the above-described electrodes may be processed by a processor or other components within the patch device, which will be described later.

Here, each of the at least one electrode in the patch device may include a flexible electrode. More specifically, referring to FIG. 5, each electrode (500) of the patch device may include at least one of a silicon adhesive (510), a fabric layer (520), an adhesive layer (530), and a flexible electrode (540). The electrode (500) may have a multilayer structure, allowing it to maintain a constant mechanical interlocking state with the user's body. That is, the electrode (500) may remain in close contact with the user's body and maintain the mechanical coupling even during user activity or movement. More specifically, the electrode (500) may be designed such that the electrode is wrapped with fabric and silicone adhesive. For example, the silicone adhesive may be Silbione, but it is not limited thereto.

A fabric layer (520) may be provided on the top portion of the electrode (500), and the fabric layer (520) may remain in a non-sticky state. This may help maintain smooth integration with other components or materials within the patch device.

The silicon adhesive (510) may bind the entire electrode (500), including the fabric layer (520), adhesive layer (530), and flexible electrode (540). For example, the silicon adhesive (510) may be applied in a liquid state before curing and absorbed through the adhesive layer (530) up to the top of the fabric layer (520). After application, the silicon adhesive (510) may be cured and maintain integration with the components within the electrode (500). Silicone may be a material with excellent flexibility, durability, and biocompatibility, and may protect internal materials from environmental changes (e.g., temperature, pressure, humidity, etc.). For example, the silicon adhesive (510) may be in a gel or membrane form and may have viscosity, allowing continuous adhesion to the skin or similar surfaces and maintaining adhesion even when surface deformation occurs due to user movement. Specifically, the silicon adhesive (510) may be integrated with the adhesive layer (530), expanding the contact area with the body and distributing contact pressure uniformly. For example, the adhesive layer (530) may be made of nonwoven fabric. In another example, the adhesive layer (530) may be made of TPU (thermoplastic polyurethane) or silicone. The adhesive layer (530) may be made of materials that provide mechanical coupling and electrical stability for signal sensing while being attached to the user's body, and is not limited to specific materials.

The adhesive layer (530) may also be provided with a predefined pattern considering its attachment to the user's body. In a specific example, the adhesive layer (530) may include a kirigami pattern, although it is not limited thereto. The kirigami pattern may allow the adhesive layer (530) to conform to the user's body and increase mechanical coupling. More specifically, adhesive strength may increase at the folding sections of the adhesive layer (530) according to the kirigami pattern. Without a pattern, areas of detachment may occur during contraction or expansion of the user's body, weakening mechanical coupling. In contrast, a kirigami pattern allows the adhesive layer (530) to continuously maintain adhesion during body movement, preserving mechanical coupling.

Moreover, micro-spaces created by the kirigami pattern may enable ventilation between the adhesive layer (530) and the user's body, allowing sweat to evaporate while the patch device is worn. If sweat does not evaporate, adhesion may decrease, but the ventilation effect of the kirigami pattern helps maintain adhesion and thus mechanical coupling. Additionally, the ventilation may improve user comfort while wearing the patch.

That is, the predefined pattern of the adhesive layer (530) may maintain tight contact with the skin despite decreased adhesion due to body movement, thereby enhancing mechanical coupling and comfort.

In one embodiment, the adhesive layer (530) may have a predefined pattern such as the aforementioned kirigami pattern. When the user's body expands or contracts, the adhesive layer (530) may deform accordingly and maintain mechanical coupling with the surface.

Specifically, when the user's body expands, the kirigami pattern may allow the adhesive layer (530) to expand and increase the surface area for adhesion. Conversely, when the user's body contracts, the adhesive layer (530) may contract and increase adhesive strength at the contact areas to maintain mechanical coupling.

Additionally, the silicon adhesive (510) may closely conform to microscopic curvatures of the skin surface, maintaining strong adhesion and friction even when the contact surface changes due to user movement, keeping the adhesive layer (530) fixed. The adhesive layer (530), located at the bottom, may attach directly to the skin or surface, and the flexible electrode (540) may be provided within it. The adhesive layer (530) may ensure close contact with the user's body and maintain attachment based on adhesive strength. As previously described, the adhesive layer (530) may integrate with the silicon adhesive (510), enhancing adhesion and maintaining bonding during user movement. The flexible electrode (540) in the adhesive layer (530) may have a serpentine (or S-curve) pattern. The serpentine pattern may be an S-shaped curve, providing greater elasticity than linear electrodes. Therefore, even when the patch device is subjected to stretching or compression due to user activity or environmental change, mechanical coupling can be maintained and electrical connectivity preserved. Additionally, stress may not be concentrated due to the curved pattern, improving durability. Mechanical deformation may occur in the electrode (500) based on user movement. For example, deformation may occur when the skin to which the electrode is attached stretches, compresses, bends, or twists. The serpentine pattern may allow the electrode to expand or compress accordingly, maintaining mechanical coupling and providing stability for signal sensing. That is, the flexible electrode (540) with a serpentine pattern may improve mechanical coupling and electrical stability for biometric signal acquisition.

As described above, the electrode (500) in the patch device may include a silicon adhesive (510), a fabric layer (520), and an adhesive layer (530), along with a flexible electrode (540) having a serpentine pattern, thereby improving the accuracy of biometric signal acquisition. For example, the patch device may have a flexible shape based on the silicon adhesive (510), allow smooth integration with other components via the fabric layer (520), attach to the user's body through a separate adhesive layer (530), and acquire stable electrical signals while maintaining mechanical coupling via the flexible electrode (540) with a serpentine pattern. In one example, a flexible PCB electrode with the above structure may be manufactured using laser cutting or other methods to form the patch device, but it is not limited to specific forms.

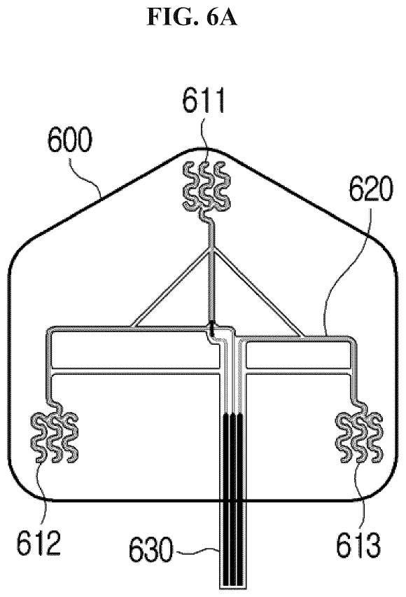

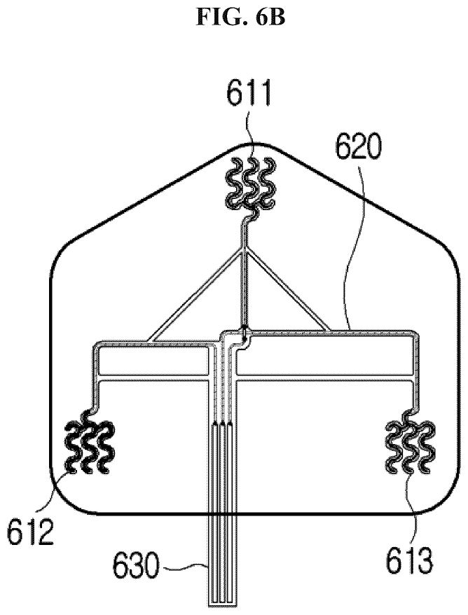

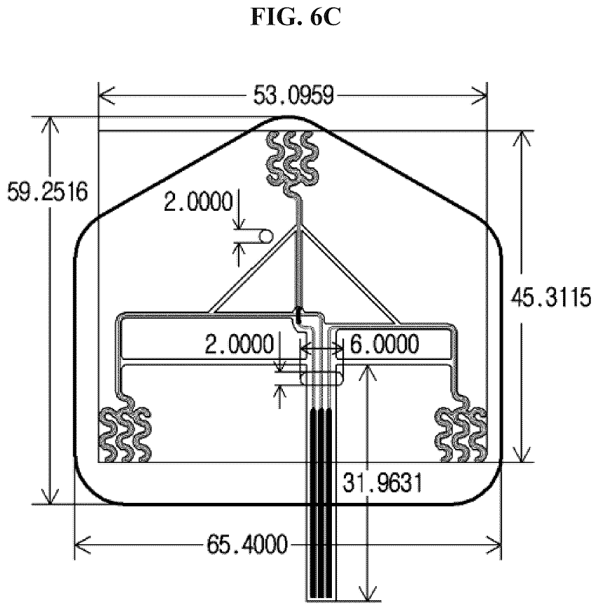

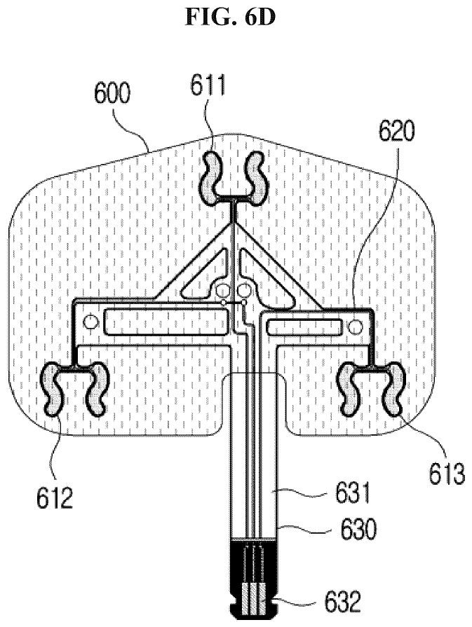

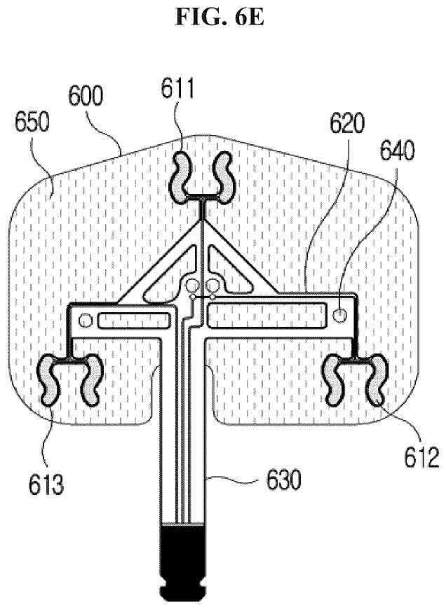

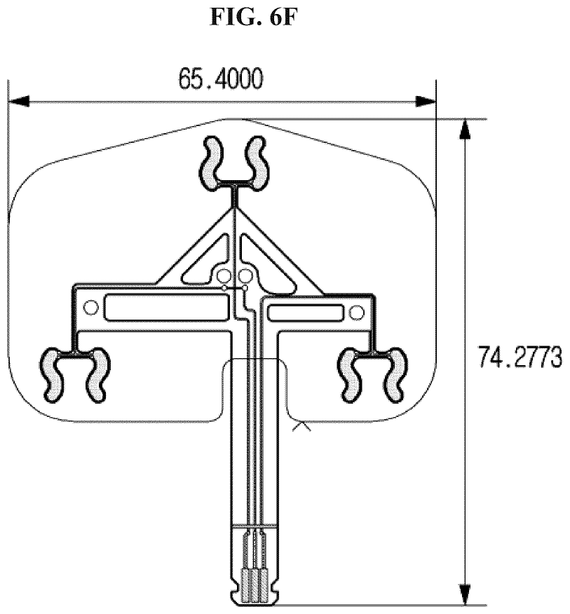

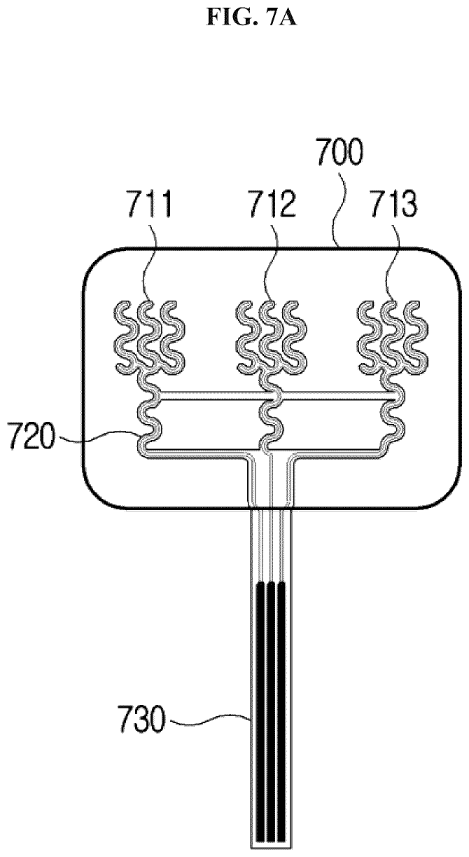

FIGS. 6A, 6B, 6C, 6D, 6E and 6F are diagrams illustrating an electrode of the patch device according to an embodiment of the present disclosure.

Referring to FIGS. 6A, 6B and 6C, the patch device (600) may include three electrodes (611, 612, 613). These three electrodes (611, 612, 613) may have the same structure as the flexible electrode described in FIG. 5. That is, each of the three electrodes (611, 612, 613) may include a silicon adhesive, a fabric layer, an adhesive layer, and a flexible electrode with a serpentine pattern. For example, the patch device (600) having the three electrodes (611, 612, 613) may be a heart patch for measuring ECG, although it is not limited thereto. In one embodiment, the flexible electrodes with a serpentine pattern corresponding to the three electrodes (611, 612, 613) may be ENIG (Electroless Nickel Immersion Gold) electrodes composed of two layers—electroless nickel and immersion gold—formed on a printed circuit board (PCB), where gold may contact the user's body. However, this embodiment is not limited thereto. Electroless nickel may be formed on copper to prevent direct contact between copper and gold, and the immersion gold layer, formed as a thin layer on the nickel, may be coated on the nickel surface based on the immersion process. Immersion gold may exhibit excellent electrical conductivity, and the gold in contact with the user's body may improve the accuracy of biometric signal acquisition. Additionally, the three electrodes (611, 612, 613) may be connected to a copper conductive layer (620) or other conductive layers. The biometric signals acquired from the user via the three electrodes (611, 612, 613) may be transmitted through the conductive layer (620) to a connector (630). The rear side of the connector (630) may be composed of ENIG electrodes, and the front side may include a flat flexible cable (FFC), forming a signal transmission path for the biometric signals sensed by the patch device (600).

In one embodiment, the patch device (600) may be sized to be attached to the region near the user's heart, functioning as a heart patch. Specifically, the patch device (600) may be configured to have dimensions of 53(L)×45(M) mm, although this is merely an example and not limiting. Additionally, a polyimide layer may be formed between the conductive layers (620) in the patch device (600) to enhance mechanical performance. In another example, the patch device (600) may include a hole into which a microphone is inserted. The microphone may be a device that converts sound into electrical energy. For example, the microphone may acquire heart sound information and deliver it to the patch device (600). Furthermore, the patch device (600) may include other components related to sensing biometric signals and is not limited to a specific form.

Referring to FIGS. 6D, 6E and 6F, the basic structure of the patch device (600) may be the same as in FIGS. 6A, 6B and 6C. However, in FIGS. 6D, 6E and 6F, the shapes of the three electrodes (611, 612, 613) may be configured differently from those in FIGS. 6A, 6B and 6C. Specifically, each of the three electrodes (611, 612, 613) may be of a serpentine pattern, for example, a pattern such as illustrated in FIGS. 6D, 6E and 6F. However, the present disclosure is not limited thereto, and other types of patterns may also be employed.

Referring again to FIGS. 6D, 6E and 6F, a connection portion (630) may include a signal transmission portion(631), which is an area constituted by electrodes within a flexible region, and an external fastening portion (632), which is constituted by electrodes within a rigid (support) region and can be connected to an external device. Specifically, the signal transmission portion (631) may be a part that transmits signals acquired from the three electrodes (611, 612, 613) within the connection portion (630) and may be located in the flexible region; the signal transmission portion (631) may be formed of electrodes. An end of the signal transmission portion (631) may be connected to the external fastening portion (632), and the external fastening portion (632) may be a part that allows the electrodes and circuits within the patch device (600) to be connected, through external coupling, to a drive unit of the patch device (600) or to an external device. Because the external fastening portion (632) must be coupled to terminals of the drive unit within the patch device (600) or to terminals of an external device, it may be formed of a rigid material. Specifically, if the external fastening portion (632) were formed of a flexible material, connection to the drive unit of the patch device (600) (or to an external device) might not be smooth. For example, if the region of the external fastening portion (632) bends or folds, the portion that must be joined to the terminal of the external device may fail to join, causing defects at the terminal. In view of the foregoing, the external fastening portion (632) may be configured as a fixed structure in the support (rigid) region and formed of a rigid material, thereby enabling connection to an external device without defects. In contrast, although the signal transmission portion (631) includes electrodes, it may not be a region directly coupled to the drive unit or to terminals of an external device, and thus may be flexible. The signal transmission portion (631) may bend so as to move the external fastening portion (632) to a position where it contacts the drive unit or an external terminal, thereby supporting the connection of the external fastening portion (632).

Furthermore, the patch device (600) may be provided with at least one fixing groove (640). At least one fixing groove (640) may be a portion configured so that a substrate on which the electrodes inside the patch device (600) are formed can be fixed to medical tape (650) (or another flexible material). For example, the medical tape (650) or other flexible materials may be materials having elasticity and therefore may readily contract and relax. As another example, when the medical tape (650) or other flexible materials are minutely displaced, measured values (or measurements) may differ from patch device to patch device. In view of the foregoing, there is a need to align the medical tape (650) and the internal substrate of the patch device (600) to a constant position, and the fixing groove (640) may be a portion that aligns the substrate and the medical tape to a fixed position. Accordingly, the patch device (600) may be provided with at least one fixing groove (640). Based on the fixing groove (640), the substrate and the medical tape (650) may be sequentially overlapped, thereby enabling fabrication of patch devices (600) of the same form. Other matters may be the same as in FIGS. 6A, 6B and 6C.

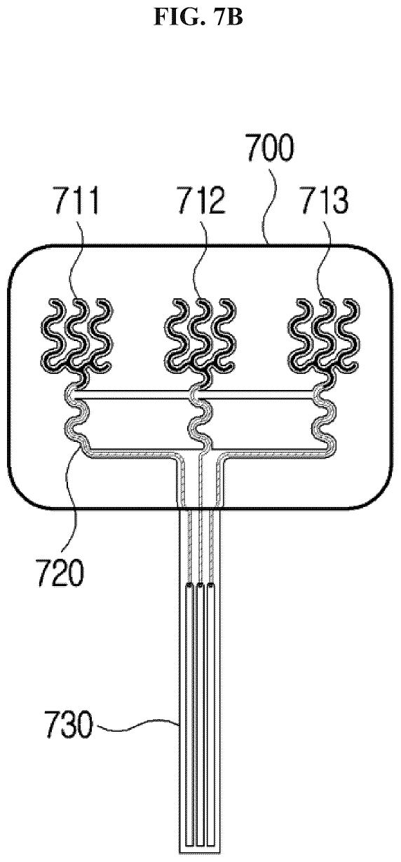

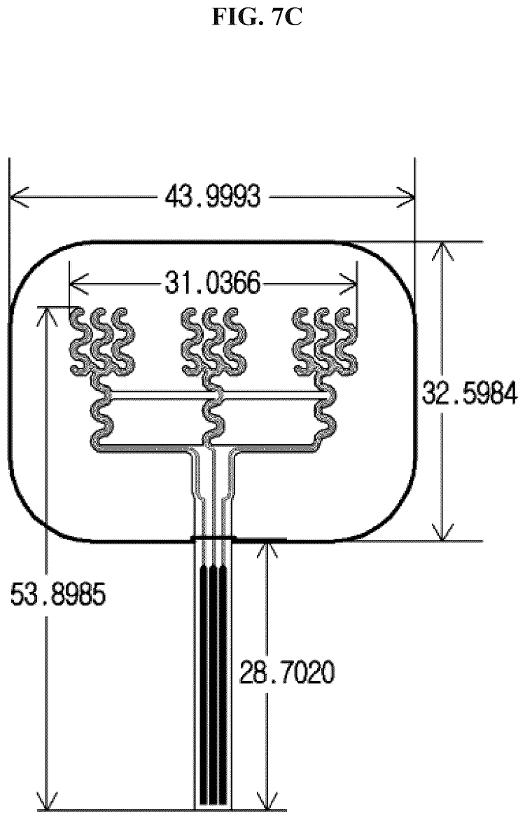

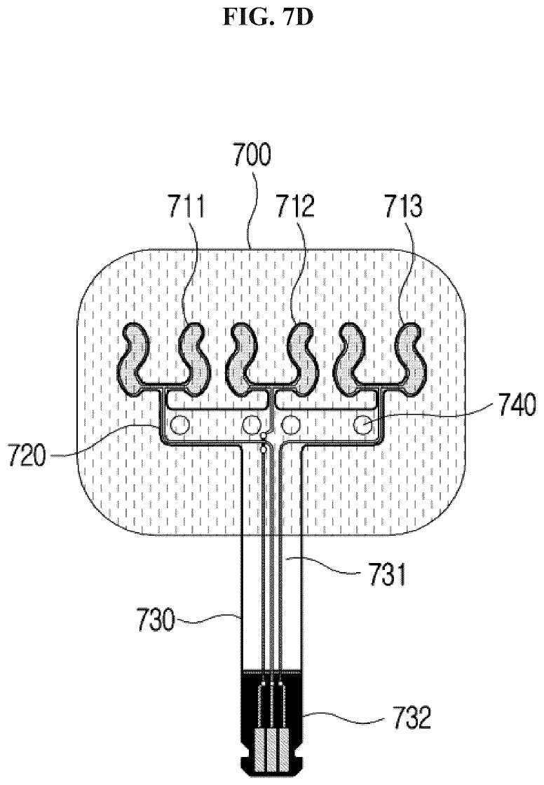

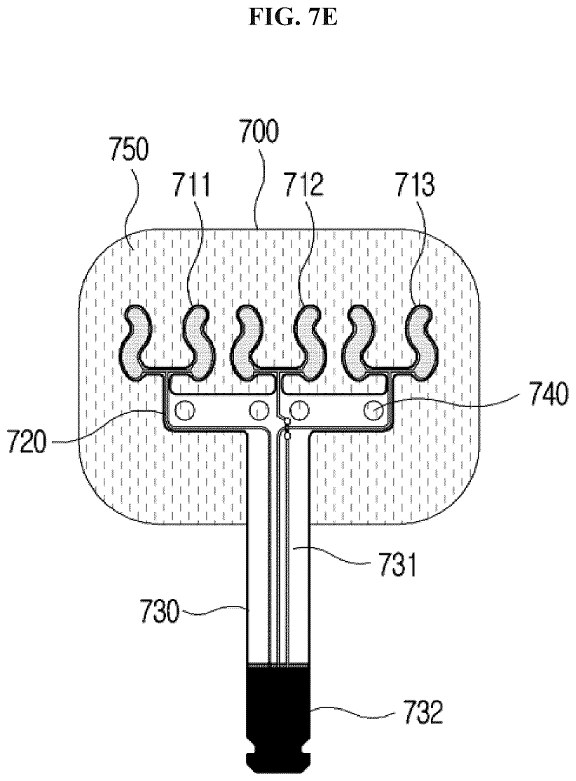

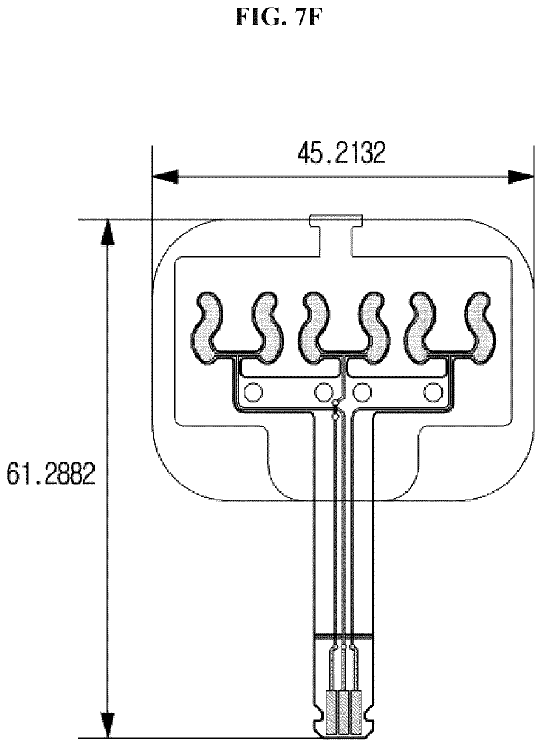

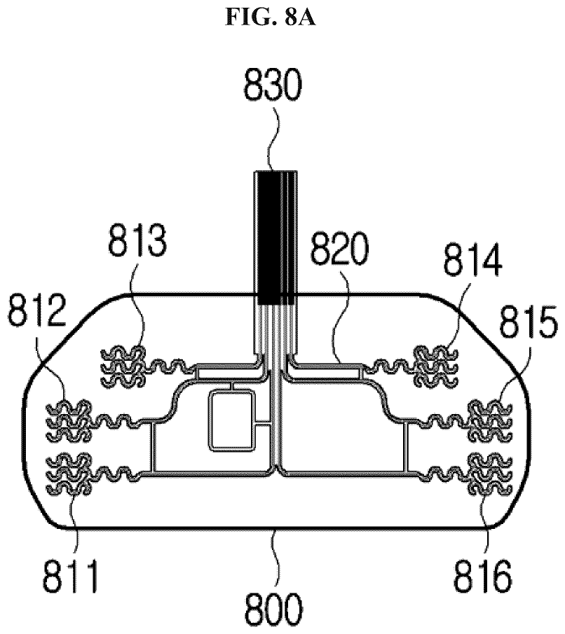

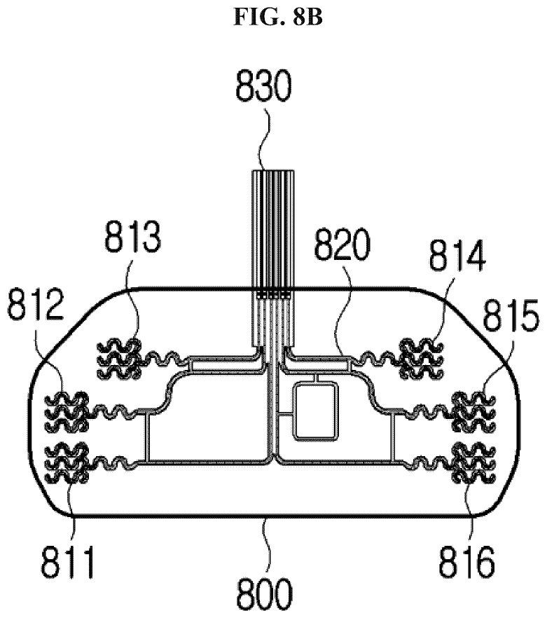

FIGS. 7A, 7B, 7C, 7D, 7E and 7F are a diagram illustrating another electrode configuration of the patch device according to an embodiment of the present disclosure.

Referring to FIGS. 7A, 7B and 7C, the patch device (700) may include three electrodes (711, 712, 713). These three electrodes (711, 712, 713) may have the same structure as the flexible electrode described in FIG. 5. That is, each of the three electrodes (711, 712, 713) may include a silicon adhesive, a fabric layer, an adhesive layer, and a flexible electrode with a serpentine pattern. For example, the patch device (700) having the three electrodes (711, 712, 713) may be a muscle patch for measuring EMG, although it is not limited thereto. In one embodiment, the flexible electrodes with a serpentine pattern corresponding to the three electrodes (711, 712, 713) may be ENIG (Electroless Nickel Immersion Gold) electrodes composed of two layers-electroless nickel and immersion gold-formed on a printed circuit board (PCB), where gold may contact the user's body. In another embodiment, the electrode may be configured based on an electroplating plateable material (e.g. gold) is electrochemically deposited. Specifically, gold ions may be reduced on the electrode to form solid gold. In this case, the gold deposited by the electroplating may be formed in such a manner as to be attached to the user's body. It should be understood that the electrode may also be configured using other methods, and is not limited to the above-described embodiment.

Electroless nickel may be formed on copper to prevent direct contact between copper and gold, and the immersion gold layer, formed as a thin layer on the nickel, may be coated on the nickel surface based on the immersion process. Immersion gold may exhibit excellent electrical conductivity, and the gold in contact with the user's body may improve the accuracy of biometric signal acquisition. Additionally, the three electrodes (711, 712, 713) may be connected to a copper conductive layer (720) or other conductive layers. The biometric signals acquired from the user via the three electrodes (711, 712, 713) may be transmitted through the conductive layer (720) to a connector (730). The rear side of the connector (730) may be composed of ENIG electrodes, and the front side may include a flat flexible cable (FFC), forming a signal transmission path for the biometric signals sensed by the patch device (700).

In one embodiment, the patch device (700) may be sized to be attached to the user's body as a muscle patch. Specifically, the patch device (700) may be configured to have dimensions of 31(L)×9(M) mm, although this is merely an example and not limiting. Additionally, a polyimide layer may be formed between the conductive layers (720) in the patch device (700) to enhance mechanical performance.

Referring to FIGS. 7D, 7E and 7F, the basic structure of the patch device (700) may be the same as in FIGS. 7A, 7B and 7C. However, in FIGS. 7D, 7E and 7F, the shapes of the three electrodes (711, 712, 713) may be configured differently from those in FIGS. 7A, 7B and 7C. Specifically, each of the three electrodes (711, 712, 713) may have a serpentine pattern, for example, a pattern such as illustrated in FIGS. 7D, 7E and 7F. However, the present disclosure is not limited thereto, and other types of patterns may also be employed.

Referring again to FIGS. 7D, 7E and 7F, a connection portion (730) may include a signal transmission portion (731), which is an area constituted by electrodes within a flexible region and is configured to transmit signals, and an external fastening portion (732), which is constituted by electrodes within a rigid (support) region and can be connected to an external device. Specifically, the signal transmission portion (731) may be a part that transmits signals acquired from the three electrodes (711, 712, 713) within the connection portion (730) and, within the flexible region, the signal transmission portion (731) may be formed of electrodes. The external fastening portion (732) may be located at an end of the signal transmission portion (731), and the external fastening portion (732) may be a part that allows the electrodes and circuits within the patch device (700) to be connected, through coupling with an external device, to a drive unit of the patch device (700) or to the external device. Because the external fastening portion (732) must be coupled to the drive unit of the patch device (700) or to an external device, it may be formed of a rigid material. Specifically, if the external fastening portion (732) were formed of a flexible material, connection to an external device might not be smooth. For example, if the region of the external fastening portion (732) bends or folds, the portion that must be joined to a terminal of the external device may fail to join, causing defects at the terminal. In view of the foregoing, the external fastening portion (732) may be configured as a fixed structure in the support (rigid) region and formed of a rigid material, thereby enabling connection to an external device without defects. In contrast, although the signal transmission portion (731) includes electrodes, it may not be a region directly coupled to the drive unit or to terminals of an external device, and thus may be flexible. Here, the signal transmission portion (731) may bend so as to move the external fastening portion (732) to a position where it contacts the drive unit or an external terminal, thereby supporting the connection of the external fastening portion (732).

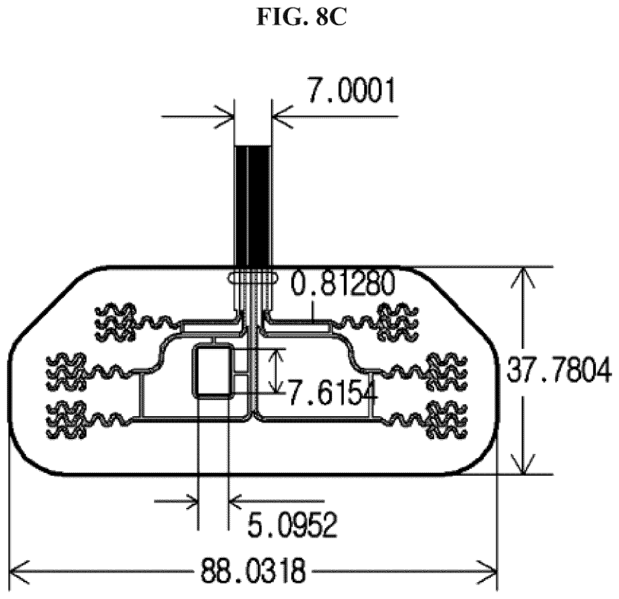

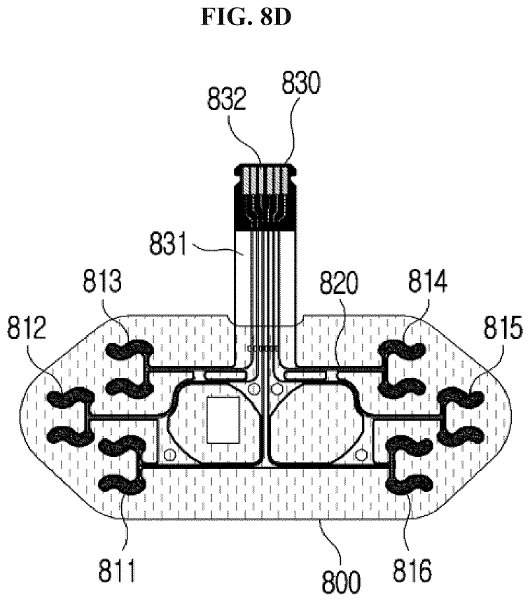

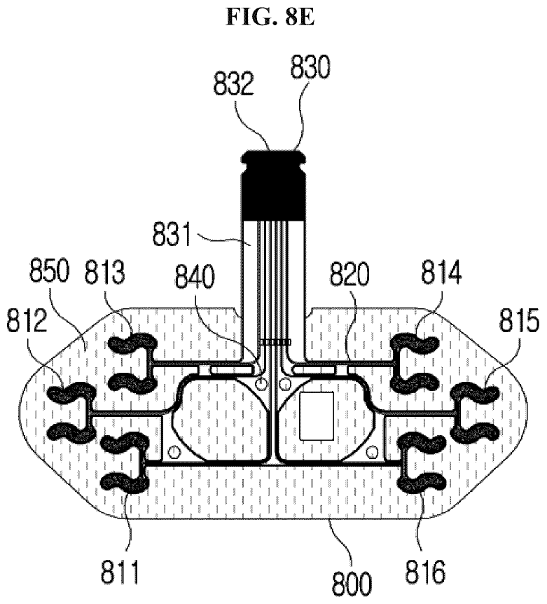

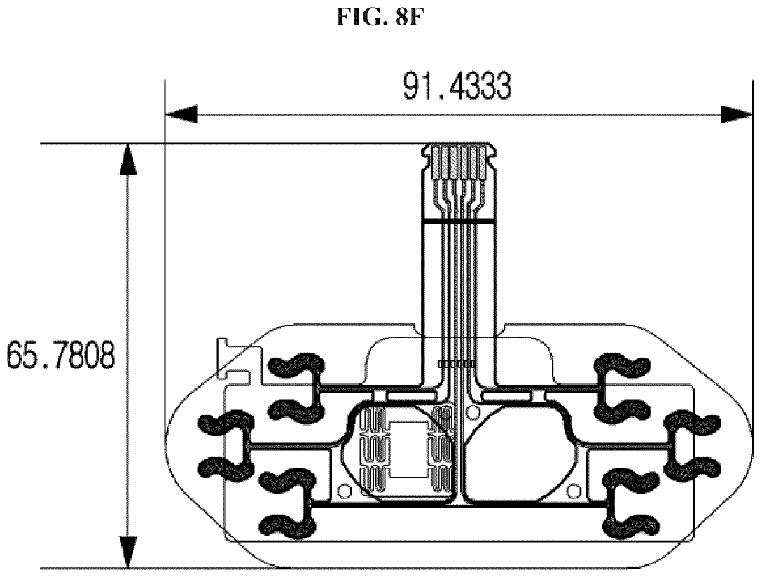

Furthermore, the patch device (700) may be provided with at least one fixing groove (740). At least one fixing groove (740) may be a portion configured so that a substrate on which the electrodes inside the patch device (700) are formed can be fixed to medical tape (750) (or another flexible material). For example, the medical tape (750) or other flexible materials may be materials having elasticity and therefore may readily contract and relax. As another example, when the medical tape (750) or other flexible materials are minutely displaced, measurement values may differ from patch device to patch device. In view of the foregoing, there is a need to align the medical tape (750) and the internal substrate of the patch device (700) to a constant position, and the fixing groove (740) may be a portion that aligns the substrate and the medical tape to a fixed position. Accordingly, the patch device (700) may be provided with at least one fixing groove (740). Based on the fixing groove (740), the substrate and the medical tape (750) may be sequentially overlapped, thereby enabling fabrication of patch devices (700) of the same form. Other matters may be the same as in FIGS. 7A, 7B and 7C. FIGS. 8A, 8B, 8C, 8D, 8E and 8F are another diagram illustrating an electrode configuration of the patch device according to an embodiment of the present disclosure.

Referring to FIGS. 8A, 8B and 8C, the patch device (800) may include six electrodes (811, 812, 813, 814, 815, 816). These six electrodes may have the same structure as the flexible electrode described in FIG. 5. That is, each of the six electrodes may include a silicon adhesive, a fabric layer, an adhesive layer, and a flexible electrode with a serpentine pattern. For example, the patch device (800) having six electrodes may be a head patch for measuring EEG, although it is not limited thereto. In one embodiment, the flexible electrodes with a serpentine pattern corresponding to the six electrodes may be ENIG (Electroless Nickel Immersion Gold) electrodes composed of two layers-electroless nickel and immersion gold-formed on a printed circuit board (PCB), where gold may contact the user's body. However, this embodiment is not limited thereto. Electroless nickel may be formed on copper to prevent direct contact between copper and gold, and the immersion gold layer, formed as a thin layer on the nickel, may be coated on the nickel surface based on the immersion process. Immersion gold may exhibit excellent electrical conductivity, and the gold in contact with the user's body may improve the accuracy of biometric signal acquisition. Additionally, the six electrodes may be connected to a copper conductive layer (820) or other conductive layers. The signals sensed through the conductive layer (820) may be transmitted to a connector (830). The rear side of the connector (830) may be composed of ENIG electrodes, and the front side may include a flat flexible cable (FFC), forming a signal transmission path for the biometric signals sensed by the patch device (800).

In one embodiment, the patch device (800) may be sized to be attached to the user's body as a head patch. Specifically, the patch device (800) may be configured to have dimensions of 76(L)×26(M) mm, although this is merely an example and not limiting. Additionally, a polyimide layer may be formed between the conductive layers (820) in the patch device (800). In another example, the patch device (800) may further include a region in which a PPG (Photoplethysmography) sensor is provided. The PPG sensor may measure blood flow variations through a light source and thus measure the aforementioned oxygen saturation. Specifically, oxygen saturation may refer to the oxygen concentration in the blood, and the PPG sensor may include a light source and a light sensor. The light sensor may measure infrared and visible light to obtain oxygen saturation information in the blood. In addition, the PPG sensor may sense blood pressure or other blood flow-related information, and is not limited to a specific type.

Referring to FIGS. 8D, 8E and 8F, the basic structure of the patch device (800) may be the same as in FIGS. 8A, 8B and 8C. However, in 8D, 8E and 8F, the shapes of the six electrodes (811, 812, 813, 814, 815, 816) may be configured differently from those in FIGS. 8A, 8B and 8C. Specifically, each of the six electrodes (811, 812, 813, 814, 815, 816) may have a serpentine pattern, for example, a pattern such as illustrated in 8D, 8E and 8F. However, the present disclosure is not limited thereto, and other types of patterns may also be employed.

Referring again to 8D, 8E and 8F, a connection portion (830) may include a signal transmission portion (831), which is an area constituted by electrodes within a flexible region and is configured to transmit signals, and an external fastening portion (832), which is constituted by electrodes within a rigid (support) region and can be connected to an external device. Specifically, the signal transmission portion (831) may be a part that transmits signals acquired from the six electrodes (811, 812, 813, 814, 815, 816) within the connection portion (830) and, within the flexible region, the signal transmission portion (831) may be formed of electrodes. The external fastening portion (832) may be located at an end of the signal transmission portion (831), and the external fastening portion (832) may be a part that allows the electrodes and circuits within the patch device (800) to be connected, through coupling, to a drive unit of the patch device (800) or to an external device. Because the external fastening portion (832) must be coupled to the drive unit of the patch device (800) or to an external device, it may be formed of a rigid material. Specifically, if the external fastening portion (832) were formed of a flexible material, connection to an external device might not be smooth. For example, if the region of the external fastening portion (832) bends or folds, the portion that must be joined to a terminal of the external device may fail to join, causing defects at the terminal. In view of the foregoing, the external fastening portion (832) may be configured as a fixed structure in the support (rigid) region and formed of a rigid material, thereby enabling connection to an external device or to the drive unit of the patch device (800) without defects. In contrast, although the signal transmission portion (831) includes electrodes, it may not be a region directly coupled to the drive unit or to terminals of an external device, and thus may be flexible. Here, the signal transmission portion (831) may bend so as to move the external fastening portion (832) to a position where it contacts the drive unit or an external terminal, thereby supporting the connection of the external fastening portion (832), details of which will be described later.

Furthermore, the patch device (800) may be provided with at least one fixing groove (840). At least one fixing groove (840) may be a portion configured so that a substrate on which the electrodes inside the patch device (800) are formed can be fixed to medical tape (850) (or another flexible material). For example, the medical tape (850) or other flexible materials may be materials having elasticity and therefore may readily contract and relax. As another example, when the medical tape (850) or other flexible materials are minutely displaced, measurement values may differ from patch device to patch device. In view of the foregoing, there is a need to align the medical tape (850) and the internal substrate of the patch device (800) to a constant position, and the fixing groove (840) may be a portion that aligns the substrate and the medical tape to a fixed position. Accordingly, the patch device (800) may be provided with at least one fixing groove (840). Based on the fixing groove (840), the substrate and the medical tape (850) may be sequentially overlapped, thereby enabling fabrication of patch devices (800) of the same form. Other matters may be the same as in FIGS. 8A, 8B and 8C.

The patch devices described in FIGS. 6 through 8 may include electrodes having flexible electrodes with serpentine patterns, thereby enhancing mechanical coupling strength when attached to the user's body and maintaining stability in acquiring electrical signals.

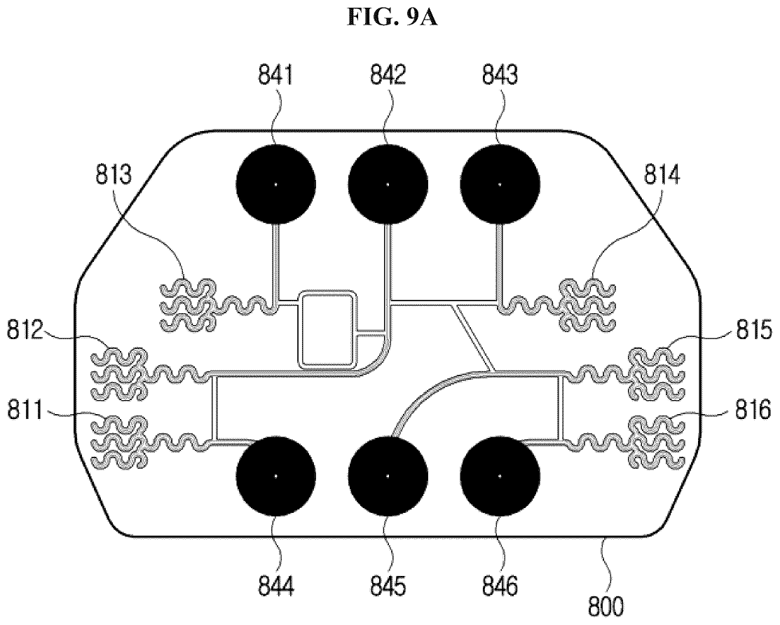

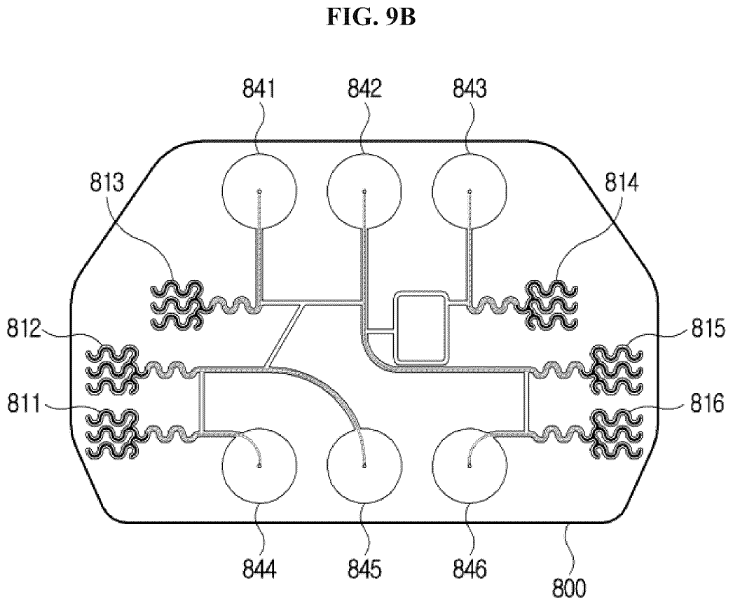

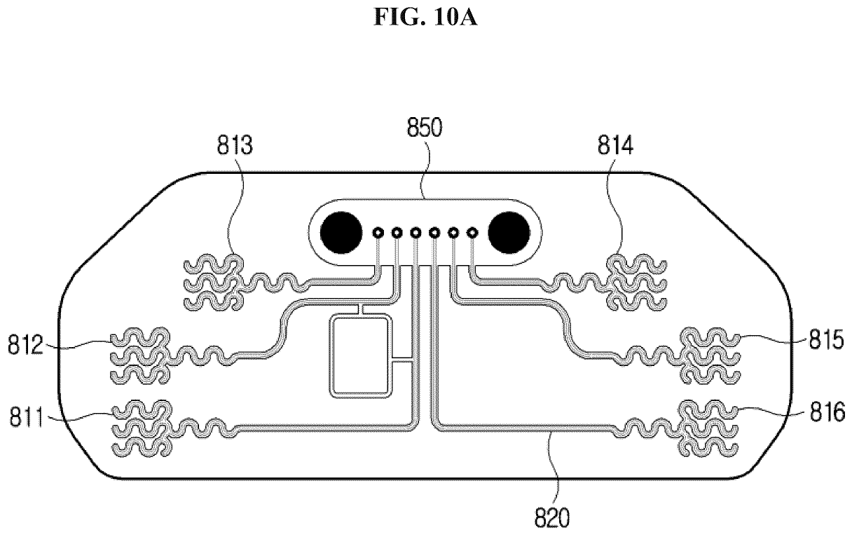

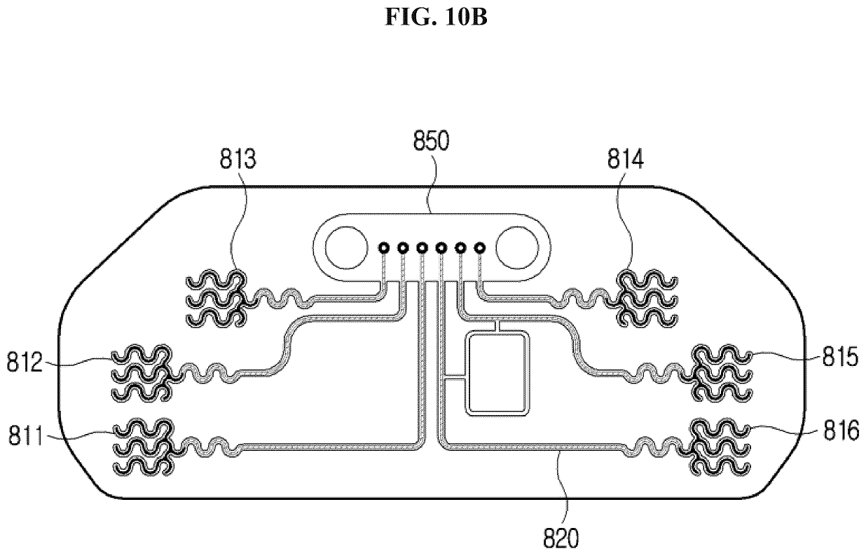

FIG. 9 and FIG. 10 are diagrams illustrating a connection version of the patch device according to an embodiment of the present disclosure. In one embodiment, FIGS. 9 and 10 describe the patch device (800) of FIG. 8 as a reference, but this is only for convenience of explanation and can equally apply to the patch devices described in FIGS. 6 and 7. However, the following description will refer to the patch device (800) of FIG. 8.

Referring to FIG. 9, the patch device (800) may be configured such that each of the six electrodes (811, 812, 813, 814, 815, 816) is connected to a respective FFC (flat flexible cable) connector (821, 822, 823, 824, 825, 826) through a conductive layer (820). Each FFC connector (821 through 826) may be connected to one of the six electrodes and transmit signals sensed from each electrode. FFC connectors can support numerous signal lines in a small space, allowing for flexible wiring. By using FFC connectors, signal transmission in narrow spaces can be performed smoothly, and patch devices can be designed more flexibly with various contact arrangements.

In one embodiment, FIG. 9 shows a structure in which three FFC connectors (841, 842, 843) are placed on the upper side and three connectors (844, 845, 846) are placed on the lower side. These FFC connectors (841, 842, 843, 844, 845, 846) may be connected to other components (or entities) within the patch device (800). The other components in the patch device may include at least one of the following: analog digital converter (ADC), processor, motion sensor, voltage regulator, voltage divider, LEDs for indicating device status, reverse voltage protection diode, ESD protection diode, wireless battery charging module, battery connecting pad, switch, and connector for firmware flashing, although not limited thereto. That is, at least one of the FFC connectors may transmit signals sensed through at least one of the electrodes to other components within the patch device (800), where the signals can be processed and utilized. In one example, the user biometric signals sensed by the six electrodes (811, 812, 813, 814, 815, 816) and transmitted via the FFC connectors may be converted into digital signal form by a processor and then transmitted to a user device through a transceiver.

On the other hand, referring to FIG. 10, the patch device (800) may be configured such that each of the six electrodes (811, 812, 813, 814, 815, 816) is connected to a pogo pin (850) through the conductive layer (820). Each of the six electrodes may be connected to a respective terminal of the pogo pin (850), and the biometric signals sensed by each electrode may be transmitted to the pogo pin (850). In one example, the pogo pin (850) may acquire signals from each of the six electrodes and may be positioned on the upper part of the patch device (800), although this is not limited thereto. Here, the pogo pin (850) may also be connected to other components within the patch device (800), such as an analog digital converter (ADC), processor, motion sensor, voltage regulator, voltage divider, LEDs for indicating device status, reverse voltage protection diode, ESD protection diode, wireless battery charging module, battery connecting pad, switch, and connector for firmware flashing, although not limited thereto. That is, the pogo pin (850) may acquire user biometric signals sensed from the six electrodes and transmit the signals to other components within the patch device (800). In one example, the signals acquired via the pogo pin (850) may be converted into digital signal form by a processor and transmitted to a user device through a transceiver.

In one embodiment, the patch device may convert the user biometric signals acquired as described above into digital signals through an ADC, and the converted digital signals may be controlled by a processor. The processor of the patch device may be a BLE (Bluetooth Low Energy) microprocessor, although this is not limited thereto. A BLE microprocessor may be a compact processor that supports low-power Bluetooth communication and may support wireless applications based on low power consumption. In one example, the patch device may operate on a low-power basis, and the above-described BLE microprocessor may be used, although not limited thereto. The processor, which integrates CPU, memory, RF transceiver, and various I/O devices into one chip, may store digital signals converted by the ADC and deliver them to an external device.

Additionally, the patch device may include a voltage regulator to supply power to the processor and other components. In one example, the voltage regulator may be a 3V voltage regulator, although this is not limiting. The voltage divider may distribute voltage supplied to the processor and for battery level monitoring. The LED may indicate the operating state of the muscle patch. For example, when the patch device is sensing signals, the LED may remain turned on, although this is not limited to the embodiment. The reverse voltage protection diode may be a diode that protects the circuit when power is connected with the wrong polarity, and the ESD protection diode may protect the patch device from electrostatic discharge (ESD).

Additionally, the patch device may include a wireless battery charging module, enabling the patch device to be wirelessly charged. The patch device may also include a battery connecting pad, which allows for wired charging. That is, the patch device may operate by being charged with power supplied externally through the charging module. Furthermore, the patch device may include a switch that controls whether the patch device is operational. The patch device may also include a connector for firmware flashing to update applications or firmware within the patch device.

In addition, a region for placing a coil may be included in the lower portion of the patch device, and wireless charging may be performed through the coil. This coil area may include a wireless charging connecting pad, through which charging power may be supplied to the patch device.

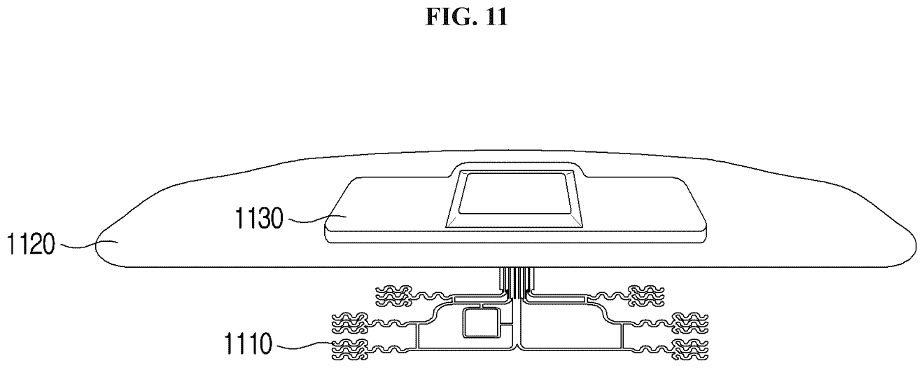

FIG. 11 is a diagram illustrating the device structure of the patch device according to an embodiment of the present disclosure. Referring to FIG. 11, the patch device may include a patch electrode (1110). The patch electrode (1110) may correspond to any one of those illustrated in FIGS. 6 through 8 and may include the above-described serpentine pattern. Additionally, the patch electrode (1110) may be configured with either an FFC connector or a pogo pin as shown in FIGS. 9 and 10, respectively, without limitation to a particular form. That is, the patch device may include a patch electrode (1110) attached to the surface of the user's body to sense signals. A medical tape layer (1120) may be located on top of the patch electrode (1110). The term “medical tape layer” is used herein for convenience and is not limited to this name. The medical tape layer (1120) may include the adhesive layer (520) and the fabric layer (530) shown in FIG. 5. A silicone adhesive portion (510) described above may also be bonded to the adhesive layer (520) and the fabric layer (530) within the medical tape layer (1120). For example, the medical tape layer (1120) may be made of a nonwoven fabric material comprising the adhesive layer (520) and the fabric layer (530). Alternatively, the medical tape layer (1120) may be made of TPU (thermoplastic polyurethane) or silicone. The medical tape layer (1120) may be made of a material that provides mechanical bonding to the user's body and ensures electrical stability for signal sensing, and is not limited to a specific type.

The medical tape layer (1120) may also have a predetermined pattern in consideration of being attached to the user's body. Specifically, a Kirigami pattern may be included. However, it is not limited to such a pattern. The Kirigami pattern may improve mechanical bonding by allowing the tape to flex and fold according to the shape of the user's body. More specifically, adhesive strength may increase in the folded parts of the medical tape layer (1120) according to the Kirigami pattern. Without such a pattern, parts of the medical tape layer (1120) may peel off due to expansion and contraction of the body, weakening mechanical bonding. In contrast, when the Kirigami pattern is applied, the adhesive surface of the medical tape layer (1120) may stay adhered to the body even under expansion or contraction, maintaining strong mechanical bonding.

Additionally, the Kirigami pattern may create micro-gaps between the user's skin and the medical tape layer (1120), allowing ventilation and evaporation of sweat even while the patch device is attached. For example, if sweat does not evaporate, the adhesive strength of the patch device may decrease due to moisture. However, with the Kirigami pattern, the ventilation feature through the micro-gaps may maintain adhesion and mechanical bonding, enhancing the ease of attachment to the body.

Thus, the predetermined pattern in the medical tape layer (1120) may help maintain skin adhesion even when the skin contracts or expands, improving bonding and usability.

In one embodiment, the medical tape layer (1120) may include the aforementioned Kirigami pattern. However, it is not limited to this. When the part of the user's body to which the medical tape layer (1120) is attached contracts or expands, the layer may be deformed according to the Kirigami pattern to maintain mechanical bonding.

More specifically, when the user's body expands, the medical tape layer (1120) may expand and increase its surface area according to the Kirigami pattern, thus maintaining adhesion. Conversely, when the user's body contracts, the medical tape layer (1120) may contract and enhance adhesive strength to maintain mechanical bonding.

Additionally, the patch electrode (1110) may include the above-described silicone adhesive portion (510), thereby enhancing mechanical bonding and improving the reliability of electrical signal sensing. The patch device may further include a control unit (1130). The control unit (1130) may process the signals sensed by the patch electrode attached to the user or transmit them to an external device. The control unit (1130) may also manage power and charging functions to control the operation of the patch device. In one example, the control unit (1130) may operate based on low power, but is not limited thereto.

The control unit (1130) in FIG. 11 may be a hardware component or a logical configuration for controlling the patch device.

For example, the control unit (1130) may include an upper and lower cover housing a control unit (e.g., mainboard) for processing sensed signals and a charging unit (e.g., charging antenna, battery), but is not limited to this configuration. That is, the control unit (1130) in FIG. 11 may take various forms for controlling the patch device and is not limited to a particular form.

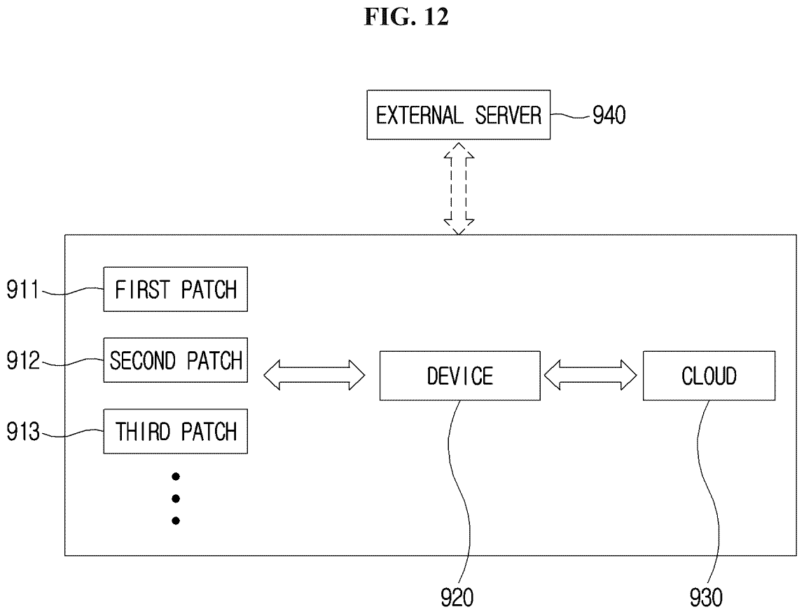

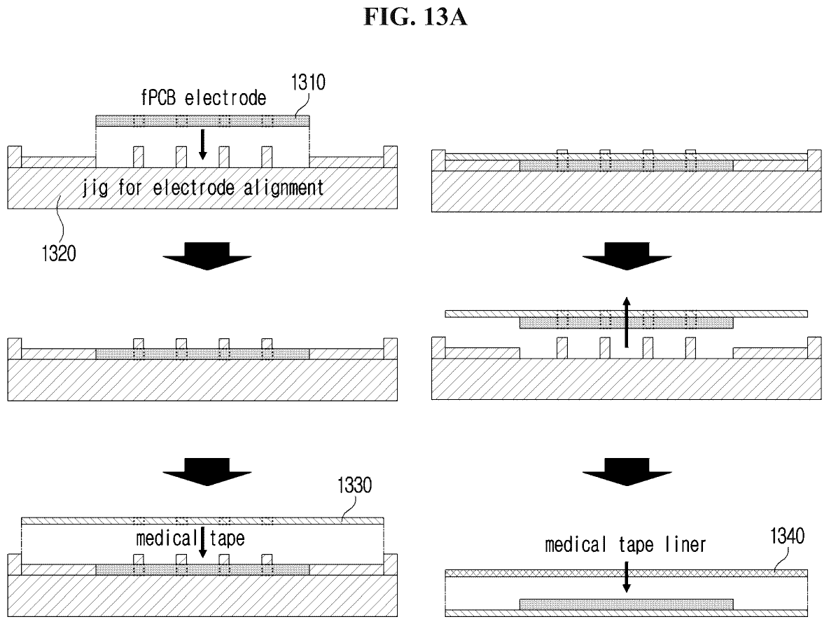

FIG. 12 is a diagram illustrating a system including at least one patch device according to an embodiment of the present disclosure.

A system (900) including at least one patch device may be considered. The system (900) may acquire biometric signals from a user through at least one patch device (911, 912, 913). In one example, the system (900) including at least one patch device (911, 912, 913) may be a user monitoring system, a sleep system, or another system that identifies user state information, but is not limited thereto.

In one embodiment, the biometric signals acquired from at least one patch device (911, 912, 913) may be transmitted to a cloud (930) through a device (920). The cloud (930) may receive the biometric signals acquired from at least one patch device (911, 912, 913) via the device (920) and perform diagnosis and analysis. Specifically, the cloud (930) may include an AI/ML learning model that performs diagnosis and analysis based on biometric signals. The cloud (930) may receive user biometric signals acquired from at least one patch device via the device (920), input the data into the AI/ML model, and derive user monitoring information as output. The AI/ML learning model may also be updated based on biometric signals acquired from the user, and is not limited to a specific form.

In another embodiment, the above-described system (900) may be connected to an external server (940). The external server (940) may provide user profile information to the system. Alternatively, the external server (940) may be a server of a medical institution or a health-related organization, and the system (900) may acquire user-related information. The information obtained from the external server (940) may be reflected in the cloud (930). Specifically, the AI/ML learning model may determine different weights for biometric signal analysis and diagnosis based on the information obtained from the external server (940). That is, the AI/ML learning model may reflect additional user-related information and perform inference based on the biometric signals acquired from the user, thereby improving the accuracy of user monitoring.

In a specific example, the system (900) may provide analysis information on the signals acquired from at least one patch device (911, 912, 913) based on AI/ML. More specifically, the biometric signals acquired from at least one patch device (911, 912, 913) may be transmitted via the device (920). The device (920) may perform inference based on embedded algorithms or monitoring learning models and derive analysis information on the signals acquired from at least one patch device (911, 912, 913). As described above, at least some of the user monitoring-related information may be input to the AI/ML model, and inference may be performed accordingly. The resulting analysis information may include user monitoring or diagnosis information.

In another embodiment, the device (920) may perform filtering, wavelet noise removal, and other preprocessing tasks, and then perform inference based on the preprocessed data. For instance, the learning model may perform inference based on rescaling or convolutional neural network (CNN) classification operations. As a result, analysis may be performed on the biometric signals acquired from at least one patch device (911, 912, 913), and monitoring and diagnosis information on the current user may be generated. Then, the device (920) may calculate periodic information based on the monitoring and diagnosis information and display the data on the device (920). In one example, the device (920) may generate score information based on the user's state monitoring and diagnosis information and display the score on the device (920). That is, the device (920) may analyze the signals acquired from at least one patch device (911, 912, 913) using AI/ML to generate state monitoring and diagnosis information. In one example, the device (920) may receive a pre-trained sleep diagnosis model from the cloud, but is not limited thereto.

In one embodiment, the above-described patch device may include at least one electrode that acquires a user's biometric signal, a conductive layer connected to each electrode, a connector that transmits the biometric signal acquired from at least one electrode to at least one entity within the patch device, and at least one entity that receives the biometric signal and transmits it to the device.

The entity may include at least one of an analog-to-digital converter (ADC), processor, motion sensor, voltage regulator, voltage divider, LED for indicating device status, reverse voltage protection diode, ESD protection diode, wireless battery charging module, battery connecting pad, switch, or connector for firmware flashing, without being limited thereto. The connector may be connected to other components (or entities) in the patch device.

Each of the at least one electrode may include a fabric layer that maintains a non-adhesive state, an adhesive layer located below the fabric layer and attached to the user's body, a flexible electrode within the adhesive layer for sensing biometric signals from the user's body, and a silicone adhesive portion that constitutes the inside of the electrode. The flexible electrode may have a serpentine pattern. For example, the silicone adhesive portion may be in a liquid state before curing and applied to the entire electrode including the fabric and adhesive layers, and then maintain a bonded state after curing. This enhances mechanical performance as previously described.

Additionally, a system including at least one patch device may be considered. The system may include at least one patch device, a device connected to and controlling the at least one patch device, and a cloud that receives biometric signals from the device and generates monitoring and analysis information, without limitation thereto.

In one embodiment, the electrode may be an ENIG (electroless nickel immersion gold) electrode comprising an electroless nickel layer formed on copper to prevent direct contact between copper and gold, and a thin layer of gold formed on nickel through immersion. The immersion gold layer may be in contact with the user's body to acquire biometric signals. Additionally, a polyimide layer may be provided between the conductive layers in the patch device to enhance mechanical performance. The connector may include at least one FFC (flat flexible cable) connector connected to each electrode or a pogo pin to which the electrodes are connected.

In one embodiment, the patch device may be a heart patch. The heart patch may include three electrodes and acquire at least one of ECG (electrocardiogram), SCG (seismocardiogram), and PCG (phonocardiogram) signals as biometric signals. The patch device may further include a microphone for acquiring heart sound information, similar to FIG. 6.

In another embodiment, the patch device may be a head patch. The head patch may include three electrodes and acquire EEG (electroencephalography) and EOG (electrooculography) signals, and use a PPG (photoplethysmography) sensor to acquire oxygen saturation information, similar to FIG. 7.

In another embodiment, the patch device may be a muscle patch. The muscle patch may include six electrodes and acquire EMG (electromyography) signals, and use a motion sensor to acquire user body movement information. The patch device may further include a PPG sensor for acquiring oxygen saturation information, similar to FIG. 8.

The patch device may acquire biometric signals and transmit them to a device, and the biometric signals may be transmitted from the device to the cloud for analysis and diagnosis. The cloud may include a learning model, and the user monitoring and analysis information may be derived as output through inference using the learning model's input, as previously described.

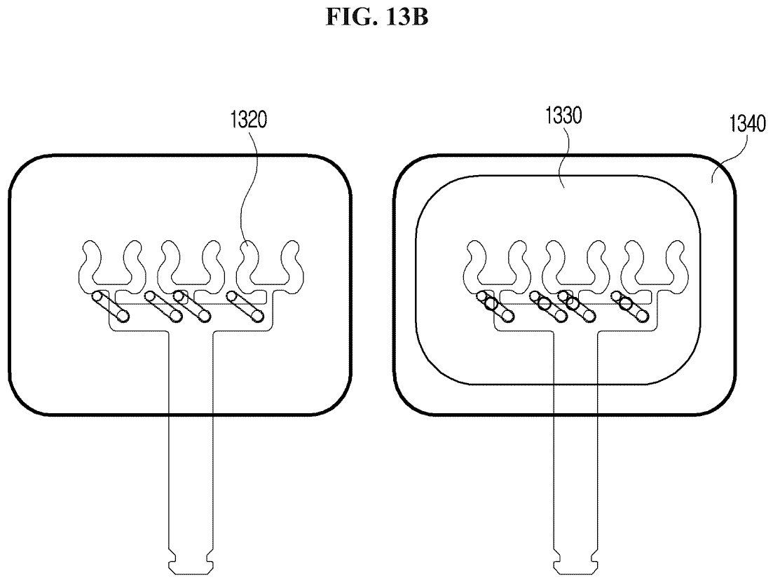

FIGS. 13A and 13B are views illustrating a method of manufacturing a flexible circuit substrate applicable to the present disclosure. Referring to FIG. 13A, a flexible circuit substrate may be formed as an fPCB (flexible printed circuit board electrode) (1310). The fPCB (1310) may constitute the electrodes included inside the above-described patch devices and may denote a substrate on which the electrodes shown in FIGS. 6a, 6b, 7a, 7b, 8a, and 8b are formed. In one example, the fPCB (1310) may be in the form in which electrodes are formed on a flexible circuit substrate and may have flexibility so as to be attachable to skin or curved surfaces. The fPCB (1310) may be of a thin and lightweight structure so as to be attachable to human skin, as described above. In addition, the fPCB (1310) may allow high-precision circuit formation, making it possible to fabricate fine electrode patterns. Here, the fPCB (1310) may be fabricated on the basis of an electrode-alignment jig (1320). Because the fPCB (1310) is a flexible circuit substrate, circuits within a flexible region may be formed while the fPCB (1310) is placed on the electrode-alignment jig (1320). The electrode-alignment jig (1320) and the fPCB (1310) may be secured on the basis of the above-described fixing grooves (640, 740, 840). That is, the fPCB (1310) may be provided with the fixing grooves (640, 740, 840) and may be coupled to the electrode-alignment jig (1320) through the fixing grooves (640, 740, 840). In this way, the fPCB (1310) placed on the electrode-alignment jig (1320) can always be fixed at the same position. Thereafter, medical tape (1330) may be coupled to the fPCB (1310) on which the electrodes have been formed, and the medical tape (1330) may likewise be fixed on the basis of the fixing grooves (640, 740, 840). For example, referring to FIG. 13B, the electrode-alignment jig (1320) and the medical tape (1330) may be fixed with reference to the fixing grooves (640, 740, 840) in the fPCB (1310). Through the foregoing, the elastic medical tape (1330) can be coupled to the fPCB (1310) at a fixed position, and after the medical tape (1330) is coupled to the fPCB (1310), the fPCB (1310) may be detached from the electrode-alignment jig (1320). Thereafter, on the side opposite to the side where the fPCB (1310) and the medical tape (1330) are coupled, a medical tape liner (1340) may be further coupled, thereby forming the above-described internal configuration of the patch device.

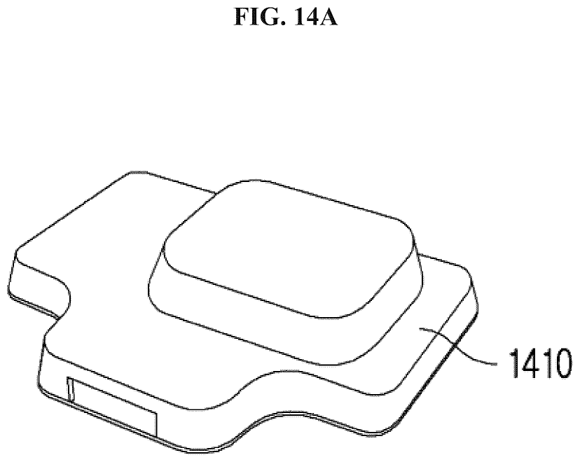

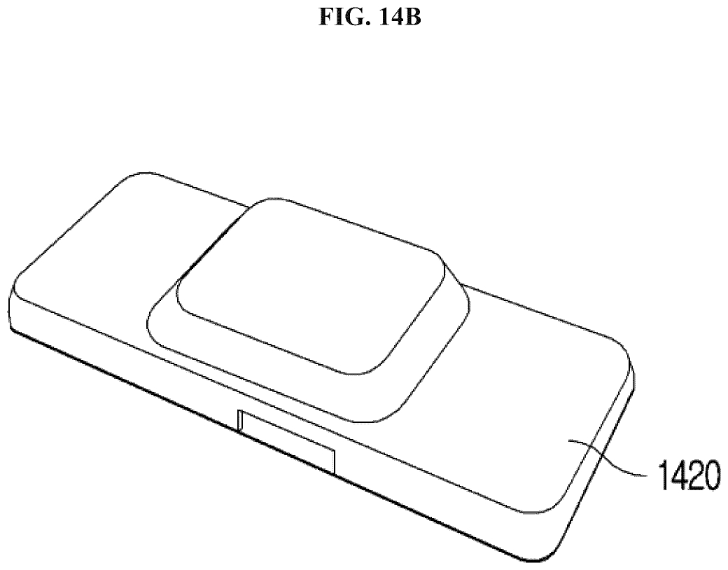

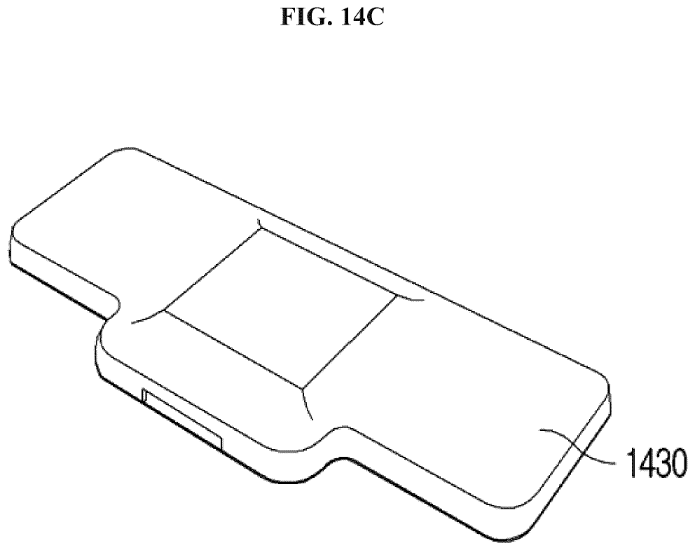

FIG. 14 illustrates configurations related to a patch device housing applicable to the present disclosure. Referring to FIG. 14, the patch device may be considered for use as the above-described heart patch, head patch, and muscle patch. Here, the patch device housing may take forms such as those shown in FIGS. 14(a), 14(b), and 14(c). However, the patch device housings(1410, 1420, 1430) of FIG. 14 are merely examples, and variations in shape may be implemented; the invention is not limited to a specific form. For convenience of explanation, the following description is given with reference to a particular patch device housing (1430) of FIG. 14, but the same may apply to the other patch device housings (1410, 1420), and the invention is not limited to a specific form. Referring to FIG. 14, at the lower side of the patch device housing (1430), there may be located a region that contacts the user's skin, namely a medical tape (or flexible region) to which the fPCB is coupled.

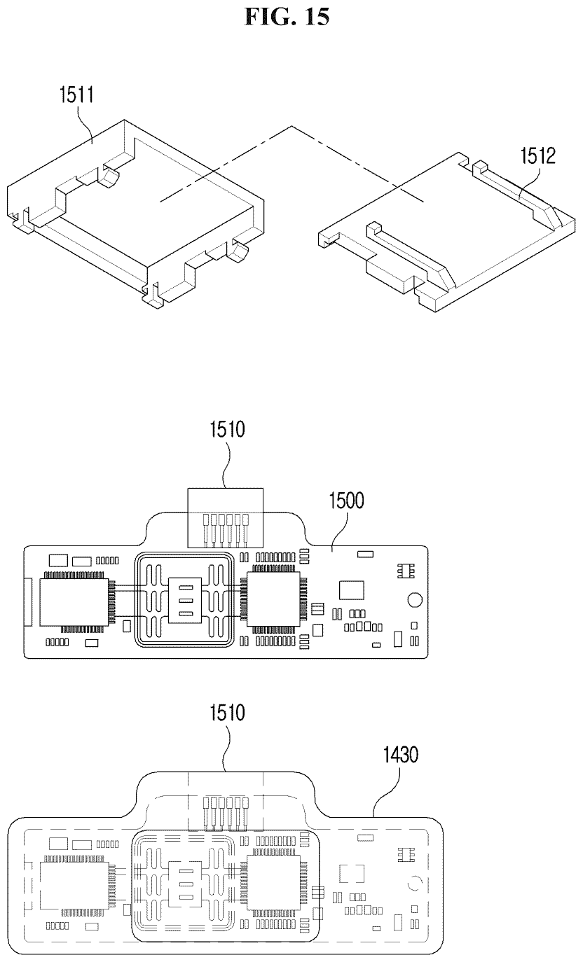

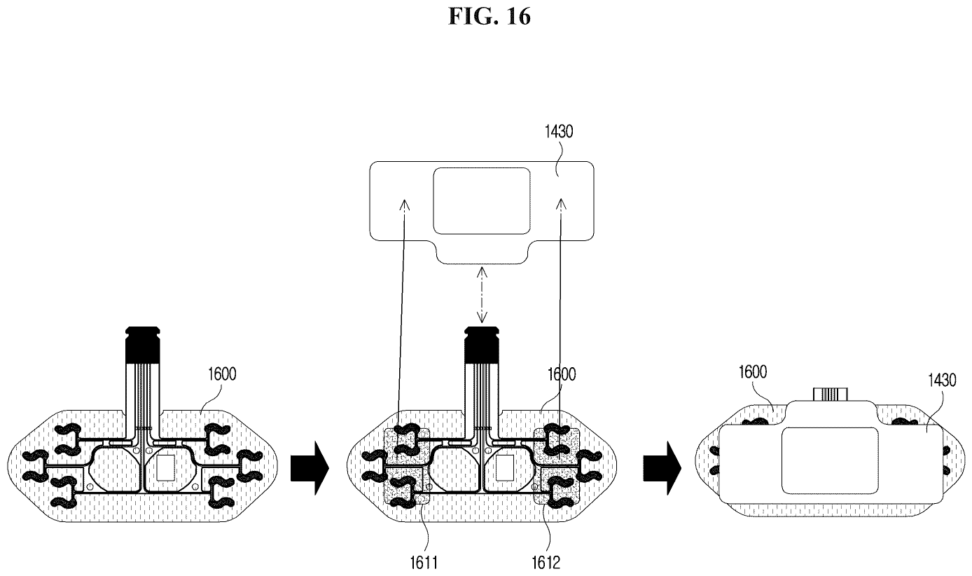

In a more specific example, FIGS. 15 and 16 are views illustrating structures of a patch device applicable to the present disclosure.

Referring to FIG. 15, within the patch device housing (1430), a drive unit (1500) related to driving of the patch device housing (1430) may be included. In one example, the drive unit may include at least one of: an electrode (electrode connector), an analog-to-digital converter (ADC), a processor, a voltage regulator, a voltage divider, LEDs for indicating device status, a reverse-voltage protection diode, an ESD (electrostatic discharge) protection diode, a wireless battery charging module, a battery connecting pad, a switch (421), and a connector for firmware flashing. For example, a signal measured by the electrodes via sensors may be converted into a digital signal through the ADC, and the converted digital signal may be controlled by the processor. The processor may be a BLE (Bluetooth Low Energy) microprocessor, but is not limited thereto. A BLE microprocessor may be a small microprocessor supporting low-power Bluetooth communication and may support wireless applications on the basis of low power. In one example, the drive unit (1500) of the patch device housing (1430) may operate on a low-power basis, and the above-described BLE microprocessor may be used, but the invention is not limited thereto. The processor may be implemented as a single chip including a CPU, a memory, an RF transceiver, and various I/O devices, and may store the digital signal converted by the ADC and transmit it to an external device. The drive unit (1500) of the patch device housing (1430) may further include a voltage regulator to supply power to the processor and other components. The voltage divider may distribute voltages for the processor supply and for monitoring the battery level, and the LEDs may indicate an operating state of the patch device housing (1430). The reverse-voltage protection diode may protect the circuit when power is connected with incorrect polarity in the head patch, and the ESD protection diode may protect the head patch from electrostatic discharge. The drive unit (1500) of the patch device housing (1430) may include a wireless battery charging module so that the drive unit (1500) can be wirelessly charged. The drive unit (1500) of the patch device housing (1430) may include a battery connecting pad so that wired charging is also possible. That is, the drive unit (1500) of the patch device housing (1430) may receive external power through a charging module, be charged, and operate. The drive unit (1500) of the patch device housing (1430) may further include a switch for controlling whether the drive unit (1500) operates. The drive unit (1500) of the patch device housing (1430) may further include a connector for firmware flashing to update an application or firmware. The drive unit (1500) of the patch device housing (1430) may include a region in which a coil is disposed so that wireless charging is performed through the coil. However, the above-described components included in the drive unit (1500) of the patch device housing (1430) are merely examples and are not limiting. Various components on a circuit board may be included in the drive unit (1500). The drive unit (1500) may be an individual device and is not limited to a specific form. In the present disclosure, it is referred to as the drive unit (1500) for convenience of explanation.

Referring to FIG. 15, in an upper portion of the drive unit (1500) of the patch device housing (1430), a coupling-fastening portion (1510) that couples with an external fastening portion of the medical tape (or flexible region) (1600) to which the fPCB is coupled may be included. Here, the coupling-fastening portion (1510) may include an upper fastening portion (1511) and a lower fastening portion (1512). The coupling-fastening portion (1510) may be configured so that the electrode of the external fastening portion of the medical tape (or flexible region) (1600) to which the fPCB is coupled is connected in a fixed manner to the electrode of the drive unit (1500). The upper fastening portion (1511) and the lower fastening portion (1512) within the coupling-fastening portion (1510) may be configured to surround the outside of the electrode of the drive unit (1500), and the electrode of the external fastening portion of the medical tape (or flexible region) (1600) to which the fPCB is coupled may be inserted into a space formed by the coupling-fastening portion (1510) and thereby be coupled to the electrode of the drive unit (1500). Through the foregoing, the medical tape (or flexible region) (1600) to which the fPCB is coupled can be connected to the drive unit (1500) within the patch device housing (1430) and can deliver measurement signals sensed while attached to the body.

In a specific example, referring to FIG. 16, the external fastening portion of the medical tape (or flexible region) (1600) to which the fPCB is coupled may be connected to the patch device housing (1430), and at the portion connected to the patch device housing (1430) there may be disposed the coupling-fastening portion (1510) of the above-described drive unit (1500). That is, the external fastening portion of the medical tape (or flexible region) (1600) to which the fPCB is coupled may be inserted into a groove of the patch device housing (1430), and may be fixed within the coupling-fastening portion (1510) located in the groove so as to be connected to the electrode of the drive unit (1500). Here, a signal transmission portion of the medical tape may bend so that the external fastening portion becomes positioned at a location where it is connected to the patch device housing (1430), thereby supporting external coupling of the external fastening portion. Thereafter, after the electrode of the external fastening portion and the electrode of the drive unit (1500) are connected, the patch device housing (1430) may be coupled to adhesive portions (1611, 1612) of the medical tape (or flexible region) (1600). In one example, the adhesive portions (1611, 1612) may be of a hook-and-loop (e.g. Velcro) type, and corresponding adhesive portions (not shown) may be provided at corresponding positions on the patch device housing (1430) to allow coupling with the hook-and-loop type; however, the invention is not limited thereto. For example, the adhesive portions (1611, 1612) of the medical tape (or flexible region) (1600) and the adhesive portions of the patch device housing (1430) may be other adhesive types capable of adhesion (or coupling), whereby the patch device housing (1430) and the medical tape (1600) may be coupled. As described above, a lower region of the medical tape (or flexible region) (1600) is attached to the body so that relevant signals can be sensed from the user.

The above-described embodiments may be at least partially implemented as a computer program and recorded on a computer-readable storage medium. The storage medium readable by a computer includes all types of storage devices in which data readable by a computer is stored. Examples of such media include ROM, RAM, CD-ROM, magnetic tape, and optical data storage devices. The computer-readable medium may also be distributed across network-connected computer systems so that code stored and executed in a distributed manner can be read by a computer. Functional programs, code, and code segments for implementing the present disclosure may be easily understood and implemented by those skilled in the art.

As described above, the present disclosure has been described with reference to the embodiments illustrated in the drawings, which are merely exemplary, and it will be understood by those skilled in the art that various modifications and variations of the embodiments are possible. However, such modifications should be regarded as falling within the technical scope of protection of the present disclosure. Therefore, the true scope of technical protection of the present disclosure should be defined to include other implementations, other embodiments, and equivalents to the claims attached hereto based on the technical spirit of the claims.

[Description of Reference Numerals]

| | |
|---|---|
| 310: Heart patch | 320: Head patch |
| 330: Muscle patch | 410: Smartphone |
| 420: Tablet | 430: Smart watch |
| 440: PC | 450: Laptop |
| 500: Electrode | 510: Silicone adhesive portion |
| 520: Fabric layer | 530: Adhesive layer |
| 540: Flexible electrode | 600: Patch device |
| 611: Electrode | 612: Electrode |
| 613: Electrode | 620: Conduction layer |
| 630: Connector | 631: Signal transmission portion of the connector |
| 632: External fastening portion | |
| 700: Patch device | 711: Electrode |
| 712: Electrode | 713: Electrode |
| 720: Conduction layer | 730: Connector |
| 731: Signal transmission portion of the connector | |
| 732: External fastening portion | |
| 800: Patch device | 811: Electrode |
| 812: Electrode | 813: Electrode |
| 814: Electrode | 815: Electrode |
| 816: Electrode | 820: Conduction layer |
| 830: Connector | 831: Signal transmission portion of the connector |
| 832: External fastening portion | |
| 841: FFC connector | |
| 842: FFC connector | 843: FFC connector |
| 844: FFC connector | 845: FFC connector |
| 846: FFC connector | 850: Pogo pin |
| 900: System | 911: Patch device |
| 912: Patch device | 920: Device |
| 930: Cloud | 940: External server |
| 1110: Patch electrode | 1120: Medical tape layer |
| 1130: Control unit | |
| 1410: Patch device housing | 1420: Patch device housing |
| 1430: Patch device housing | 1500: Drive unit |
| 1510: Coupling-fastening portion | 1511: Upper fastening portion |
| 1512: Lower fastening portion | 1600: Medical tape |
| 1611: Adhesive portion | 1612: Adhesive portion |

1. A patch device comprising:
at least one electrode configured to acquire a user biometric signal;
a conductive layer connected to each of the at least one electrode;
a connector connected to the conductive layer and configured to transmit the user biometric signal acquired from the at least one electrode to at least one entity within the patch device; and
the at least one entity configured to receive the user biometric signal and transmit the signal to a device,
wherein each of the at least one electrode comprises:
a fabric layer maintaining a non-sticky state;
an adhesive layer located below the fabric layer and attachable to a user's body;
a flexible electrode within the adhesive layer and configured to sense the user biometric signal from the user's body; and
a silicone adhesive portion coupled to the entire electrode,
wherein the flexible electrode has a serpentine pattern.

2. The patch device of claim 1,
wherein the electrode comprises at least one of electroplating and an ENIG (electroless nickel immersion gold) electrode, wherein the electroplating comprises
wherein the ENIG electrode comprises an electroless nickel layer formed on copper to prevent direct contact between the copper and gold, and an immersion gold layer formed as a thin gold layer on the nickel,
and wherein the immersion gold layer contacts the user's body to acquire the user biometric signal.

3. The patch device of claim 1,
wherein a polyimide layer for improving mechanical performance is further included between the conductive layers of the patch device,
and the connector comprises at least one flat flexible cable (FFC) connector connected to each of the at least one electrode or a pogo pin connected to the at least one electrode.

4. The patch device of claim 1,
wherein the adhesive layer in the patch device has a predetermined pattern, and when the portion of the user's body to which the adhesive layer is attached contracts or expands, the adhesive layer is deformed according to the predetermined pattern to maintain mechanical bonding with the surface of the user's body.

5. The patch device of claim 1,
wherein the predetermined pattern is a Kirigami pattern,
and when the user's body expands, the adhesive layer expands according to the Kirigami pattern, increasing its surface area, and the increased surface area adheres to the user's body to maintain mechanical bonding,
and when the user's body contracts, the adhesive layer contracts according to the Kirigami pattern, increasing the adhesive strength in the contact area with the user's body to maintain mechanical bonding.

6. The patch device of claim 1,
wherein the patch device is a heart patch comprising a plurality of electrodes,
wherein the heart patch acquires at least one signal among ECG (electrocardiogram), SCG (seismocardiogram), and PCG (phonocardiogram) as the user biometric signal through the plurality of electrodes,
and wherein, in case the patch device is the heart patch, a microphone is further included and configured to acquire heart sound information.

7. The patch device of claim 1,
wherein the patch device is a head patch comprising a plurality of electrodes,
wherein the head patch acquires EEG (electroencephalography) and EOG (electrooculography) signals through the plurality of electrodes, and acquires oxygen saturation information through a PPG (photoplethysmography) sensor.

8. The patch device of claim 1,
wherein the patch device is a muscle patch comprising a plurality of electrodes,
wherein the muscle patch acquires an EMG (electromyography) signal through the plurality of electrodes and acquires user body movement information through a motion sensor, and
wherein, in case the patch device is the muscle patch, a PPG (photoplethysmography) sensor is further included and configured to acquire oxygen saturation information.

9. The patch device of claim 1,
wherein the patch device includes at least one of a heart patch, head patch, and muscle patch, and the patch device acquires the user biometric signal and transmits it to a device,
wherein the user biometric signal is transmitted from the device to a cloud for analysis and diagnosis.

10. The patch device of claim 8,
wherein the user biometric signal is transmitted from the device to the cloud, and monitoring and analysis information is generated,
wherein the cloud includes a learning model, and the user monitoring and analysis information is provided as input to the learning model and is output through inference by the learning model.

11. A system comprising at least one patch device, the system comprising:
the at least one patch device;
a device connected to and configured to control the at least one patch device; and
a cloud configured to acquire user biometric signals from the device and generate monitoring and analysis information,
wherein each of the at least one patch device comprises:
at least one electrode configured to acquire a user biometric signal;
a conductive layer connected to each of the at least one electrode;
a connector connected to the conductive layer and configured to transmit the user biometric signal acquired from the at least one electrode to at least one entity within the patch device; and
the at least one entity configured to receive the user biometric signal and transmit the signal to the device,
wherein each of the at least one electrode comprises:
a fabric layer maintaining a non-sticky state;
an adhesive layer located below the fabric layer and attachable to a user's body;
a flexible electrode within the adhesive layer and configured to sense the user biometric signal from the user's body; and
a silicone adhesive portion coupled to the entire electrode,
wherein the flexible electrode has a serpentine pattern.
